(19) United States
(12) Patent Application Publication
(10) Pub. No.: US 2025/0129380 A1
(43) Pub. Date: Apr. 24, 2025

(54) METHOD FOR SELECTING AN IMPROVED GENETICALLY MODIFIED PLANT

(71) Applicants: Yissum Research Development Company of the Hebrew University of Jerusalem Ltd., Jerusalem (IL); The State of Israel, Ministry of Agriculture & Rural Development, Agricultural Research Organization, Rishon Lezion (IL)

(72) Inventors: Naomi ORI, Jerusalem (IL); Alon ISRAELI, Jerusalem (IL); Maya BAR, Rishon Lezion (IL)

(73) Assignees: Yissum Research Development Company of the Hebrew University of Jerusalem Ltd., Jerusalem (IL); The State of Israel, Ministry of Agriculture & Rural Development, Agricultural Research Organization, Rishon Lezion (IL)

(21) Appl. No.: 18/835,166

(22) PCT Filed: Feb. 1, 2023
(86) PCT No.: PCT/IL2023/050115

§ 371 Date: Aug. 1, 2024

Related U.S. Application Data
(60) Provisional application No. 63/267,407, filed on Feb. 1, 2022.

(51) Int. Cl. C12N 15/82; C12Q 1/6895

(52) U.S. Cl. CPC C12N 15/8282; C12N 15/8261; C12Q 1/6895; C12Q 2600/13

(57) ABSTRACT

The present invention, is directed to a method for selecting an improved genetically modified Solanum plant, the method including: determining the presence of at least one inactive allele of auxin responsive factor (ARF) 8 gene in the genome of the genetically modified Solanum plant or a part derived therefrom; and (b) selecting a genetically modified Solanum plant determined as having a genome including the at least one inactive allele of ARF8 gene, wherein the improvement is at least any one of: (i) increased yield; (ii) increased resistance to a pathogen; (iii) earlier fruit setting; and (iv) any combination of (i) to (iii), compared to a wild-type variant of said Solanum plant, thereby, selecting an improved genetically modified Solanum plant.

### CROSS-REFERENCE TO RELATED APPLICATIONS

This application claims the benefit of priority of U.S. Provisional Patent Application No. 63/267,407, titled “INCREASING YIELD STABILITY IN PLANTS”, filed 1 Feb. 2022, the contents of which are incorporated herein by reference in their entirety.

### FIELD OF INVENTION

The present invention relates to a method for improving plant yield.

### BACKGROUND

Fruits are major sources of flavor, nutrition, and fibers in the human diet and in the food industry. Fruits develop from the ovary, which contains the ovules. Following its growth and patterning during flower development, the gynoecium pauses growth until fertilization. Upon fertilization, ovules differentiate into seeds, and the surrounding maternal ovary resumes growth and develops into a fruit, a process termed fruit set. Normally, fruit set occurs only upon fertilization, and in the absence of fertilization the flower aborts. As a consequence, fruit set is compromised under non-optimal temperatures that prevent fertilization, limiting the growing season, yield and fruit quality. These limitations are likely to increase as the climate warms and heat waves become more frequent. Many lines of research have led to yield increase. However, even when the yield potential is high, unfavorable conditions can lead to severe yield loss. Genetic relaxation of the coupling between fertilization and fruit set may contribute to the realization of yield potential by increasing yield stability under unfavorable environments.

Uncoupling fruit set from fertilization results in the formation of parthenocarpic fruits, which develop without seeds. Parthenocarpic fruits can result from genetic, environmental or hormonal alterations. The signals for fruit set include auxin and other hormones produced by the embryo and/or endosperm within the developing seeds. Parthenocarpic fruits form as a result of altered auxin response; when auxin is overproduced in ovaries of transgenic plants; in response to inhibition of auxin transport; or in transgenic plants with perturbed auxin physiology. Thus, local alteration of auxin response is sufficient to promote fruit development in plants with diverse fruit biology. This has been exploited in tomato (Solanum lycopersicum) cultivation by auxin applications, which promote fruit production in cool conditions when pollination is inefficient. An alternative to auxin application could be a manipulation of the auxin response pathway, which may lead to a cheaper and more efficient relaxation of the dependence of fruit set on fertilization without the use of chemicals.

Class A Auxin Response Transcription Factors (class A ARFs) are central components of the nuclear auxin signal transduction pathway. In the presence of auxin, they activate expression of auxin-responsive genes. Conversely, in the absence of auxin, these ARFs can repress gene expression when complexed with Aux/IAA transcriptional repressors. Auxin switches class A ARFs activity from repression to activation by promoting Aux/IAA protein turnover. There are five class A ARFs in Arabidopsis and seven in tomato, and these act in a partially overlapping manner to regulate growth in various tissues. For example, Arabidopsis AtARF6, AtARF8, and AtNPH4/ARF7 could promote or inhibit hypocotyl elongation, depending on the growth conditions and genetic background. Negative feedback loops as well as inputs from other signals may contribute to the non-linear gene dosage responses in this and other contexts. In tomato, fine-tuning the activity of class A ARFs and their Aux/IAA repressor S1IAA9/ENTIRE caused a phenotypic continuum of leaf complexity.

Several class A ARF proteins affect flower and fruit development. Arabidopsis class A ARFs AtARF6 and AtARF8, which are negatively regulated by the microRNA miR167, promote growth in hypocotyls, leaves, inflorescence stems, and flower organs. Flowers of Atarf6 Atarf8 (Atarf6,8) double mutants are largely male- and female sterile, as are p35S:AtMIR167a plants that overproduce miR167 to silence both AtARF6 and AtARF8. Female sterility of these plants results from a combination of defects in stigma growth, style growth and maturation, and transmitting tract differentiation that together limit the ability of pollen to germinate, grow, and fertilize mutant ovules. Of note, Atarf8 single mutants are parthenocarpic, having excess gynoecium growth in the absence of fertilization.

All tomato class A ARFs interact physically and genetically with the Aux/IAA protein S1IAA9/ENTIRE (E). Sliaa9/entire (e) mutants make fruit in the absence of fertilization, indicating that S1ARF proteins likely regulate tomato fruit set. Indeed, altered activity of SlARF5/SlMP, S1ARF7, S1ARF8, and/or S1ARF2 led to partial parthenocarpy. Similarly, downregulation of eggplant SmARF8 led to parthenocarpy. In strawberry, loss of FvARF8 function increased fruit growth. Transgenic eggplant overexpressing ARF8 also have increased fruit growth. These discoveries in multiple species suggests that ARF8 orthologs may have a broadly conserved role in fruit set. However, it is still not clear which SlARFs are the central regulators of tomato fruits set.

There is still a great need for methods of improving plant yield, such as, but not limited to tomato, under rapidly changing and/or extreme conditions, e.g., characterizing global changes.

### SUMMARY

The present invention, in some embodiments, is based, at least in part, on the findings that several tomato Slarf8 mutant combinations more than doubled the yield under extreme temperatures. Partial reduction of SlARF8 dose resulted in increased yield stability with minimal pleiotropic effects. The increased yield resulted from several developmental effects, including an early onset of fruit set, increased number of fruit-bearing branches and increased number of flowers that set fruit.

The present invention, in some embodiments, is further based, at least in part, on the surprising findings that tomato Slarf8 mutant combinations were found to be less prone, i.e., more resistant, to pathogenic infections, as well as set fruit earlier, both compared to a control wild-type plant. These features were observed not only “under extreme temperatures”.

The inventors, therefore, suggest that altering SlARF8 dose, such as by loss of function (e.g., knockout, etc.), can be utilized for the production of a genetically modified plant, characterized by increased pathogen resistance, earlier fruit setting, as well as increased yield stability in fluctuating environments.

According to one aspect, there is provided a method for selecting an improved genetically modified Solanum plant, the method comprising: (a) determining the presence of at least one inactive allele of auxin responsivefactor (ARF) 8 gene in the genome of the genetically modified Solanum plant or a part derived therefrom; and (b) selecting a genetically modified Solanum plant determined as having a genome comprising the at least one inactive allele of ARF8 gene, wherein the improvement is at least any one of: (i) increased yield; (ii) increased resistance to a pathogen; (iii) earlier fruit setting; and (iv) any combination of (i) to (iii), compared to a wild-type variant of the Solanum plant, thereby, selecting an improved genetically modified Solanum plant.

According to another aspect, there is provided a genetically modified Solanum plant comprising any one of: at least one inactive allele of ARF8a gene, at least one inactive allele ARF8b gene, and both, wherein the genetically modified Solanum plant comprises a genome being devoid of any one of: (i) one or both alleles of ARF8a gene comprising the nucleic acid sequence: ATGAAGCTTTCAACATGGAATGGGTCCAGCAAGCTCATGA (SEQ ID NO: 29), ATGAAGCTTTCCATCAGGAATGGGTCCAGCAAGCTCATGA (SEQ ID NO: 30), or both; (ii) one or both alleles of ARF8b gene comprising the nucleic acid sequence: ATGAAGCTTTCAACATCAGAGAATGGGTCAGCAGGCTCATGA (SEQ ID NO: 31), ATGAAGCTTTCTCAGGAATGGGTCAGCAGGCTCATGAAGGAGGAGAGAAAAAGTG TTTGA (SEQ ID NO: 32), or both; or (iii) both (i) and (ii).

In some embodiments, the increased yield is under culture conditions being sub-optimal for culturing the wild-type variant of said Solanum plant.

In some embodiments, the sub-optimal conditions comprise: heat sub-optimal conditions, cold sub-optimal conditions, or both.

In some embodiments, the method further comprises a step preceding the step (a), comprising producing the genetically modified Solanum plant, wherein the producing comprises contacting a Solanum plant or a part derived therefrom with an effective amount of an agent capable of inactivating any one of the allele of ARF8 gene, a transcript thereof, a protein product thereof, and any combination thereof, in the Solanum plant or a part derived therefrom, thereby producing the genetically modified Solanum plant.

In some embodiments, the agent comprises a polynucleotide, a protein, or both, being a clustered regularly interspaced short palindromic repeats (CRISPR) system.

In some embodiments, the agent comprises at least one single guide RNA (sgRNA) configured to targeting the ARF8 gene, and a CRISPR associated (Cas) protein.

In some embodiments, the sgRNA comprises the nucleic acid sequence set forth in any one of SEQ ID Nos: 21-22, 33, and any combination thereof.

In some embodiments, the Cas protein comprises Cas9 protein.

In some embodiments, the at least allele of the ARF8 gene being inactive is: knocked out, mutated, or knocked down, wherein an mRNA transcribed therefrom, a protein product translated therefrom, or both, is inactive or absent, in the genetically modified plant.

In some embodiments, two alleles of the ARF8 gene being inactive are: knocked out, mutated, or knocked down, wherein an mRNA transcribed therefrom, a protein product translated therefrom, or both, is inactive or absent, in the genetically modified plant.

In some embodiments, the Solanum plant is characterized by having a genome comprising at least two paralogs of ARF8 gene.

In some embodiments, the at least two paralogs of ARF8 gene comprise ARF8a gene and ARF8b gene.

In some embodiments, the genetically modified Solanum plant comprises a genome comprising at least one allele of any one of ARF 8a gene, ARF8b gene, or both, being: knocked out, mutated, or knocked down, such that an mRNA transcribed therefrom, a protein product translated therefrom, or both, is inactive or absent, in the genetically modified plant.

In some embodiments, the genetically modified Solanum plant comprises a genome comprising two alleles of any one of ARF8a gene, ARF8b gene, or both, being: knocked out, mutated, or knocked down, such that an mRNA transcribed therefrom, a protein product translated therefrom, or both, is inactive or absent, in the genetically modified plant.

In some embodiments, the genetically modified Solanum plant is a Solanum lycopersicum (tomato) plant.

In some embodiments, the method further comprises a step proceeding step (b), comprising culturing the selected genetically modified Solanum plant of step (b).

In some embodiments, the culturing comprises culturing at a temperature of at least 28° C.

In some embodiments, the yield comprises at least one parameter being selected from the group consisting of: number of fruit, total yield (gr), harvest index, number of inflorescences, number of fruit per inflorescence, % of parthenocarpic fruit, and any combination thereof, of the genetically modified plant.

In some embodiments, the pathogen is selected from a fungus or a bacterium.

In some embodiments, the genetically modified Solanum plant comprises at least one inactive allele of the ARF8a gene comprising the nucleic acid sequence selected from:

(SEQ ID NO: 25)
AATTACCCCGAACTTGCCACCACAGCTGTCAACTCCACAATGTCAC;
or
(SEQ ID NO: 26)
AATTACCCCGAACTTGCCACCACAGTTTGATCTGTCAACTCCACAATGT
CAC.

In some embodiments, the genetically modified Solanum comprises at least one inactive allele of the ARF8b gene comprising the nucleic acid sequence:

(SEQ ID NO: 27)
CTCTGGATCTGTCAACTCCACA.

In some embodiments, the genetically modified Solanum plant comprises: (i) at least one inactive allele of the ARF8a gene comprising the nucleic acid sequence set forth in SEQ ID NO: 25 and at least one inactive allele of the ARF8b gene comprising the nucleic acid sequence set forth in SEQ ID NO: 27; or (ii) at least one inactive allele of the ARF8a gene comprising the nucleic acid sequence set forth in SEQ ID NO: 26 and at least one inactive allele of the ARF8b gene comprising the nucleic acid sequence set forth in SEQ ID NO: 27.

In some embodiments, both alleles of any one of: the ARF8a gene, the ARF8b gene, and both, are inactive in the genetically modified Solanum plant disclosed herein.

In some embodiments, the genetically modified Solanum plant comprises: (i) two inactive alleles of the ARF8a gene comprising the nucleic acid sequence set forth in SEQ ID NO: 25 and two inactive alleles of the ARF8b gene comprising the nucleic acid sequence set forth in SEQ ID NO: 27; or (ii) two inactive alleles of the ARF8a gene comprising the nucleic acid sequence set forth in SEQ ID NO: 26 and two inactive alleles of the ARF8b gene comprising the nucleic acid sequence set forth in SEQ ID NO: 27.

In some embodiments, the genetically modified Solanum plant disclosed herein is characterized by: (i) increased yield; (ii) increased resistance to a pathogen; (iii) earlier fruit setting; and (iv) any combination of (i) to (iii), compared to a wild-type variant of the Solanum plant.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1K include images and graphs showing fruit phenotypes of Slarf8a Slarfb mutants. (1A-1D) Representative photographs of cut, self-fertilized fruits of the indicated genotypes. Slarf8ab—Slarf8a Slarf8b. Scale bar: 2 cm. (1E) Quantification of the fruit diameter of cut, self-fertilized fruits of the indicated genotypes. (n=)—number of fruits quantified. P-values represent differences from the wild-type, as determined by the Dunnett test. (1F) Quantification of the percentage of seed bearing (orange) and seedless (green) fruits from the indicated genotypes. (n=)—number of fruits analyzed. (1G-1J) Representative photographs of fruits of the indicated genotypes with or without pollination (+/−P, respectively), showing the fertilization-independent fruit set of Slarf8a Slarf8b (Slarf8ab). Panel H shows the senescent remains of an unpollinated wild-type flower. Scale bars: 2 cm. (1K) Quantification of the percentage of fruit set in the indicated genotypes and treatments. Slarf8ab—Slarf8a Slarf8b. − p, unpollinated; +p, pollinated. (n=)—number of flowers analyzed.

FIGS. 2A-2C include micrographs and graphs showing phenotypes of larf8a Slarf8b (Slarf8ab) flowers. (2A) Ovary weight of the indicated genotypes and stages of ovary development. The flower-bud stages were classified in sequential developmental stages according to bud and gynoecia size, opening of the sepals, color of the petals and opening of the flowers. Bars represent the SE of at least three biological replicates. Statistically significant differences according to student t-test are indicated. (2B-2C) Scanning electron microscope image of the stigma from wild-type (2B) and Slarf8a Slarf8b (Slarf8ab, 2C) flowers. Scale Bar: 100 μm.

FIGS. 3A-3K include images and graphs showing effect of mutations in Slarf8 genes on yield of plants grown in controlled hot conditions. Plants were grown in a controlled greenhouse under 34° C. Day/28° C. night temperatures. (3A-3E) Mature plants at the end of the experiment, fruits of a single representative plant and a representative cut fruit from plants of each of the indicated genotypes. Scale bars: 10 cm (whole plants), 2 cm (fruits). (3F-3J) Quantification of the total number of fruits (3F), total yield in grams (3G), harvest index: total yield/plant weight (3H), number of fruit-bearing branches per plant (3I) and the number of fruits per fruit-bearing branch (3J) in the indicated genotypes. (n=)—number of quantified plants or inflorescences. P-values represent differences from the wild-type, as determined by the Dunnett test. (3K) Quantification of the percentage of natural parthenocarpy of fruits from the indicated genotypes. The orange color represents fruits with seeds and the green color represents parthenocarpic, seedless fruits. (n=)—number of fruits analyzed. Genotypes are abbreviated as follows: 8a —Slarf8a; 8a 8b/+—Slarf8a Slarf8b/+; 8ab—Slarf8a Slarf8b.

FIGS. 4A-4J include images and graphs showing effect of mutations in Slarf8 genes on yield of plants grown in controlled cold conditions. Plants were grown in a controlled greenhouse under 16° C. day/10° C. night temperatures. (4A-4D) Mature plants at the end of the experiment, fruits of a single representative plant and a representative cut fruits from the indicated genotypes. Scale bars: 10 cm (whole plants), 2 cm (fruits). (4E-4I) Quantification of the total number of fruits (4E), total yield in grams (4F), harvest index: total yield/plant weight (4G), number of fruit-bearing branches per plant (4H) and the number of fruits per fruit-bearing branch (4I) in the indicated genotypes. (n=)—number of plants or inflorescences quantified. P-values represent differences from the wild-type, as determined by the Dunnett test. (4J) Quantification of the percentage of natural parthenocarpy in the indicated genotypes. The orange color represents fruits with seeds and the green color represents parthenocarpic, seedless fruits. (n=)—number of fruits quantified. Genotype abbreviation is as in FIG. 3.

FIGS. 5A-5K include images and graphs showing the effect of mutations in Slarf8 genes on yield of plants grown in ambient heat-stress conditions. Plants were grown in a net-house in the soil in the summer under field conditions, with no temperature control, during which they experienced several hours of temperature above 40° C. every day for several weeks. (5A-5E) Fruits of a single representative plant of the indicated genotypes. Scale bars: 10 cm. (5F-5K) Quantification of the total number of fruits (5F), total yield in grams (5G), harvest index: total yield/plant weight (5H), number of fruit-bearing branches per plant (5I) number of fruits per fruit-bearing branch (5J) and days to anthesis of the first flower (5K) in the indicated genotypes. (n=)—number of plants or inflorescences quantified. P-values represent differences from the wild-type, as determined by the Dunnett test. (5J) Genotype abbreviation is as in FIG. 3.

FIGS. 6A-6D include graphs showing increased and earlier fruit set in Slarf8 mutants. (6A-6B) Number of flowers per inflorescence (6A) and number of fruits on the same inflorescences of the indicated genotypes. (n=)—Number of inflorescences quantified. P-values represent differences from the wild-type, as determined by the Dunnett test. (6C-6D): Quantification of the total number of fruits per plant in the indicated genotypes and time points under heat conditions in the controlled heat (6C) and controlled cold (6D) experiments. Number of plants for each genotype (n=, 6C-6D): wild-type: 17, 7-14; Slarf8a/+ Slarf8b/+: 9; Slarf8a: 8, Slarf8a 8b/+: 14, 4-12; and Slarf8ab: 11, 6-19. P values indicate differences from the wild-type, as determined by the Dunnett test.

FIGS. 7A-7C include a graph and illustrations showing spatial expression patterns of tomato class A S1ARF genes in the ovary. (7A) Normalized counts of tomato S1ARF genes in wild-type gynoecia five days before anthesis, as assayed by RNAseq using two biological replicates. Replicate values were normalized by the size of the libraries. (7B) Cartoon showing the five ovary tissues. Red: pericarp, green: columella, brown: ovule, purple: septum and orange: placenta. (7C) Relative expression of S1ARF encoding genes in the different ovary tissues at anthesis. Data obtained from (Shinozaki et al., 2018).

FIGS. 8A-8C include sequences, schematic genes representation, and graphs showing CRISPR/Cas9-generated alleles of SlARF8A and SlARF8B. (8A) Sequences of Slarfa and Slarf8b alleles. The sequences of the guide RNAs are shown in red. Dashed lines indicate deleted sequences and a blue letter indicates an insertion. Numbers above the sequence indicate the position relative to the ATG start codon (See also materials and methods). (8B) Scheme of the SlARF8A and S1ARF8B genes. Arrowheads indicate the locations of the guide RNAs used for CRISPR/Cas9 to generate the mutant alleles. Mutant diagrams indicate the predicted protein resulting from translational stop positions after reading frame shifts. (8C) RNAseq and qRT-PCR analysis of SlARF8A and S1ARF8B mRNA expression in S2 gynoecia of wild-type and Slarf8a Slarf8b double mutants (Slarf8ab). In the RNAseq graph, average counts of two biological replicates normalized by the size of the libraries are shown. In the qRT-PCR graph, expression was measured relative to the EXP reference gene. Error bars represent the SE of at least three biological replicates, each containing at least 3 plants. Asterisks indicate statistically significant differences by student t-test, *P<0.05.

FIGS. 9A-9U include images showing Fruit phenotypes of single, double and triple Slarf mutants. (9A-9U) Representative fruits of the indicated genotypes. Cut fruits are shown for all genotypes and whole fruits for a subset of the genotypes. Slarf19ab is a Slarf19a Slarf19b double mutant; Slarf8ab is a Slarf8a Slarf8b double mutant; /+ stands for a heterozygote. In cases where variable fruit shapes were observed for a specific genotypes, several fruits are shown. Scale bars: 2 cm.

FIGS. 10A-10J include images and graphs showing SlARF8A and S1ARF8B promote plant and leaf growth partially redundantly. (10A) Two-month-old mature plants of the indicated genotypes. Slarf8ab—double Slarf8a Slarf8b mutant. Scale bar: 10 cm. (10B) Quantification of plant height in the genotypes shown in 10A. (n=)—number of plants. quantified. Different letters indicate differences by the Tukey-Kramer multiple comparison statistical test, P<0.05. (10C-10F) Mature 5^{th} leaves of the indicated genotypes. Scale bars: 2 cm. White arrowheads point to primary or intercalary leaflets and red arrowheads to missing leaflets in the intercalary domain. (10G-10H) Quantification of the leaf length (blue, left) and width (green, right) (10G) and the number of leaflets (10H) in the indicated genotypes. (n=)—number of leaves quantified. Different letters indicate differences by the Tukey-Kramer multiple comparison statistical test, P<0.05. (10I) Quantification of hypocotyl length in one-month-old plants of the indicated genotypes. (n=)—number of plants analyzed. P values indicate differences from the wild-type, calculated using the Dunnett test. (10J) One-month-old plants of the indicated genotypes. The hypocotyl is marked by a white line and a blue arrow. Li; leaf number one, etc. Scale bars: 2 cm.

FIG. 11 includes graph showing the effect of Slarf8ab on the expression of selected genes. qRT-PCR analysis of the expression of selected over-expressed and under-expressed genes, as indicated, in S2 and S3 gynoecia of wild-type (blue) and Slarf8a Slarf8b double mutants (8ab, red). Expression was measured relative to the EXP reference gene. P-values represent differences, as determined by Student's t-test (n=at least 3 biological replicates, each containing at least 5 gynoecia from different plants).

FIGS. 12A-12B include micrographs showing ovaries of wild-type and Slarf8a Slarf8b double mutants. (12A-12B) Gynoecia of a wild-type plant (12A) and a Slarf8a Slarf8b (Slarf8ab, 12B) plant at the S3-4 stage, 2-3 days before anthesis. Blue arrowheads point to the stigma, which is shown at a higher magnification at the top right corner. Scale bars: bottom: 2 mm, inset: 0.5 mm.

FIGS. 13A-13C include graphs and images showing plant height, fruit size, and timing of fruit production in Slarf8a and Slarf8b mutant combinations under heat stress conditions. (13A) Quantification of plant height of the indicated genotypes at the end of the heat experiment. Plants were 120 days old. (n=)—number of plants quantified. Different letters indicate statistically differences by the Tukey-Kramer multiple comparison statistical test, P<0.05. (13B) Average single fruit weight was calculated by dividing the total yield of each plant by the number of fruits. (n=)—number of plants quantified. Asterisks indicate statistically significant differences by the Dunnett test compared to the wild-type, ***P<0.001. (13C) Wild-type and Slarf8a Slarf8b (Slarf8ab) plants at the day of planting (top) and after 18 days under hot conditions, showing the early fruit set phenotype of the slarf8ab double mutants.

FIGS. 14A-14E include graphs and images showing plant height, fruit size, and timing of fruit production in Slarf8a and Slarf8b mutant combinations under cold stress conditions. (14A) Quantification of plant height in the indicated genotypes at the end of the cold experiment. Plants were 150 days old. (n=)—Number of plants quantified. Different letters indicate statistically significant differences by the Tukey-Kramer multiple comparison statistical test, P<0.05. (14B) Average single fruit weight was calculated by dividing the total yield of each plant by the number of fruits. (n=)—Number of plants analyzed. P values indicate differences from the wild-type, determined by the Dunnett test. (14C) Wild-type and slarf8ab plants after 120 days in cold conditions, showing the early fruit set phenotype of slarf8ab double mutants. (14D-14E) Mature leaves of the indicated genotypes after prolonged growth in cold conditions. Leaves were photographed from plants at the end of the experiment. Scale bars: 2 cm.

FIGS. 15A-15E include graphs and images showing the effect of mutations in Slarf8 genes on yield of plants grown under ambient heat-stress conditions. (15A-15B) Quantification of the total number of fruits (15A) and total yield (15B), in the indicated genotypes. Bars represent the SE of at least three biological replicates that were planted randomly in the field. Different letters indicate differences by the Tukey-Kramer multiple comparison statistical test, P<0.05. Similar results were obtained in an additional experiment. (15C-15E) Fruits of a single representative plant of the indicated genotypes. Scale bars: 2 cm.

FIGS. 16A-16B include graphs showing the effect of Slarf8 mutations on the number and weight of red and green fruits under ambient heat stress. Quantification of the total number of red and green fruits (16A) and total yield of red and green fruit (16B), in the indicated genotypes, abbreviated as in FIG. 3.

FIG. 17 includes a graph showing pollen viability of Slarf8 mutants. Quantification of the pollen viability percentage in pollen from flower buds of wild-type (WT), Slarf8a (8a) and Slarf8ab (8ab) plants, collected two days post anthesis from plants grown under either normal or heat conditions. (n=)—Number of flower buds analyzed for each genotype. P-values indicate differences from the wild-type, as determined by Dunnett test.

FIGS. 18A-18C include an image and vertical bar graphs showing that SlARF8 genotypes are disease resistant. (18A-18B) M82 tomato plants of different SlARF8 mutant genotypes were infected with 4-day old Botrytis cinerea mycelia. Disease was monitored after 4 days. (18C) SlARF8b mutants were infected with 104 Oidium neolycopersici spores. Disease was monitored after 10 days. Lesion areas/disease coverage were measured using ImageJ. Statistically significant differences were assessed using a two tailed t-test, N=40, *p<0.05, **p<0.01 (18B), or a one-way ANOVA with a Tukey post-hoc test, N=10, p<0.008 (18C).

FIGS. 19A-19D includes graphs showing that SlARF8 genotypes have stronger immune responses. (19A-19B) M82 tomato plants of different S1ARF8 mutant genotypes were challenged with the fungal elicitor EIX. (19C-19D) S1ARF8 mutants were challenged with the bacterial elicitor flg22. 19A and 19C: Total reactive oxygen species (ROS) produced, expressed in relative luminescent units (RLU). 19B and 19D: Time course of ROS production. Statistically significant differences were assessed using a two tailed t-test with Welch's correction, N=24, *p<0.05, **p<0.01, ns=non-significant.

FIGS. 20A-20F include vertical bar graphs showing that SlARF8 genotypes maintain disease resistance under extreme temperature conditions. M82 tomato plants of different SlARF8 mutant genotypes, grown in the above indicated temperatures, were infected with 4-day old B. cinerea mycelia (20A-20C), or injected with 106 CFU of X. euvesicatoria (20D-20F). Disease was monitored after 4 days, by measuring lesion area for B. cinerea, or plating serial dilutions of macerated tissue and quantifying bacterial load for X. euvesicatoria. Statistically significant differences were assessed using a one-way ANOVA with a Tukey post-hoc test, A: N≥37, p<0.022, B: N≥12, p<0.032, C: N≥10, p<0.05, D: N≥12, p<0.0067, E: N≥12 p=ns, F: N≥8, p<0.042. Resistances in temperate conditions were retained in most extreme temperatures.

### DETAILED DESCRIPTION

### Methods of Use

According to one aspect, there is provided a method for selecting an improved genetically modified Solanum plant: comprising: (a) determining the presence of at least one inactive allele of auxin responsive factor (ARF) 8 gene in the genome of the genetically modified Solanum plant or a part derived therefrom; and (b) selecting a genetically modified Solanum plant determined as having a genome comprising the at least one inactive allele of ARF8 gene, wherein the improvement is: (i) increased yield; (ii) increased resistance to a pathogen; (iii) earlier fruit setting; or (iv) any combination of (i) to (iii), compared to a wild-type variant of said Solanum plant.

According to one aspect, there is provided a method for selecting a genetically modified plant belonging to the Solanaceae family and being characterized by providing increased yield compared to a wild-type variant of the plant belonging to the Solanaceae family when cultured under heat conditions being sub-optimal for culturing the wild-type variant of the plant belonging to the Solanaceae family.

According to one aspect, there is provided a method for selecting a genetically modified plant belonging to the Solanaceae family and being characterized by providing increased yield compared to a wild-type variant when cultured under heat conditions being sub-optimal for culturing a wild-type variant of the Solanum plant.

According to one aspect, there is provided a method for selecting a genetically modified plant belonging to the Solanaceae family and being characterized by having increased resistance to a pathogen compared to a wild-type variant of the plant belonging to the Solanaceae family.

According to one aspect, there is provided a method for selecting a genetically modified plant belonging to the Solanaceae family and being characterized by setting fruit earlier compared to a wild-type variant of the plant belonging to the Solanaceae family.

According to one aspect, there is provided a method for selecting a genetically modified plant belonging to the Solanaceae family and being characterized by any combination of: (i) providing increased yield compared to a wild-type variant when cultured under heat conditions being sub-optimal for culturing a wild-type variant of the plant belonging to the Solanaceae family; (ii) having increased resistance to a pathogen compared to a wild-type variant of the plant belonging to the Solanaceae family; and (iii) setting fruit earlier compared to a wild-type variant of the plant belonging to the Solanaceae family.

In some embodiments, a plant belonging to the Solanaceae family is a Solanum plant. In some embodiments, a plant belonging to the Solanaceae family is a Capsicum plant.

The terms “Solanum” and “Solanaceae” are used herein interchangeably and refer to any plant of the family of Solanaceae, such as, but not limited to plants of the Solanum genus.

According to another aspect, the method comprises: (a) determining the presence of at least one inactive allele of Auxin responsive factor (ARF) 8 gene in the genome of the genetically modified Solanum plant or a part derived therefrom; and (b) selecting a genetically modified Solanum plant determined as having a genome comprising the at least one inactive allele of ARF8 gene.

In some embodiments, increased yield is under abiotic stress, e.g., sub-optimal temperature, such as for culturing a wild-type variant of a Solanum plant.

In some embodiments, increased yield is under temperature stress. In some embodiments, increased yield is under heat stress, cold stress, or a combination thereof.

In some embodiments, sub-optimal conditions comprise: heat sub-optimal conditions, cold sub-optimal conditions, or both, as disclosed herein.

In some embodiments, the method further comprises a step comprising producing the genetically modified Solanum plant. In some embodiments, the producing step precedes step (a). In some embodiments, the method comprises providing a genetically modified Solanum plant.

In some embodiments, producing comprises contacting a Solanum plant or a part derived therefrom with an effective amount of an agent capable of at least inactivate any one of an allele of ARF8 gene, a transcript thereof, a protein product thereof, and any combination thereof, in a Solanum plant or a part derived therefrom, thereby producing a genetically modified Solanum plant.

In some embodiments, an agent as disclosed herein comprises a polynucleotide, a protein, or both, being a clustered regularly interspaced short palindromic repeats (CRISPR) system.

In some embodiments, an agent comprises at least one single guide RNA (sgRNA) configured to targeting, or being capable of hybridizing with a nucleic acid sequence of ARF8 gene, and a CRISPR associated (Cas) protein.

In some embodiments, the method further comprises a step proceeding step (b), comprising culturing the selected genetically modified Solanum plant of step (b).

According to another aspect, there is provided a method for increasing yield of a genetically modified Solanum plant, the method comprising culturing a genetically modified Solanum plant under heat conditions, wherein the genetically modified Solanum plant is characterized by having a genome comprising at least one allele of an Auxin responsive factor (ARF) 8 paralog gene being inactive.

According to another aspect, there is provided a method for increasing yield of a genetically modified Solanum plant, the method comprising culturing a genetically modified Solanum plant under cold conditions, wherein the genetically modified Solanum plant is characterized by having a genome comprising at least one allele of an Auxin responsive factor (ARF) 8 paralog gene being inactive.

In some embodiments, a Solanum plant, comprises a plurality of ARF8 gene paralogs.

According to another aspect, there is provided a method for increasing yield of a genetically modified Solanum plant, the method comprising culturing a genetically modified Solanum plant under heat conditions, wherein the genetically modified Solanum plant is characterized by having a genome comprising at least one allele of any one of ARF 8a gene, ARF8b gene, or both, being inactive.

In some embodiments, yield comprises at least one parameter being selected from: number of fruit, total yield (gr), harvest index, number of inflorescences, number of fruit per inflorescence, % of parthenocarpic fruit, or any combination thereof.

Methods for determining yield of a plant are common and would be apparent to one of skill in the art. None-limiting examples of methods for determining number of fruit, total yield (gr), harvest index, number of inflorescences, number of fruit per inflorescence, % of parthenocarpic fruit, etc., are exemplified herein.

In some embodiments, increase or increasing is at least 5%, at least 15%, at least 25%, at least 50%, at least 75%, at least 90%, at least 95%, at least 100%, at least 200%, at least 350%, at least 500%, at least 750%, or at least 1,000% increase, compared to a control, or any value and range therebetween. Each possibility represents a separate embodiment of the invention. In some embodiments, increase or increasing is 5-100%, 15-150%, 25-200%, 50-600%, 75-400%, 90-700%, 95-850%, 100-1,200%, or 1-500%, compared to a control. Each possibility represents a separate embodiment of the invention.

In some embodiments, earlier comprises at least 1 day, 2 days, 3 days, 5 days, 7 days, 14 days, or 28 days, before a wild-type variant of the plant belonging to the Solanaceae family, or any value and range therebetween. Each possibility represents a separate embodiment of the invention. In some embodiments, earlier comprises 1-28 days, 2-30 days, 3-60 days, 1-25 days, 7-42 days, 3-44 days, or 2-50 days, before a wild-type variant of the plant belonging to the Solanaceae family. Each possibility represents a separate embodiment of the invention.

In some embodiments, heat conditions are sub-optimal for culturing a wild-type variant of a Solanum plant.

As used herein, the term “sub-optimal” encompasses any conditions known to one of skill in the art to deviate from conditions suitable for or customarily applied to a Solanum plant so as to obtain crop. In some embodiments, a wild-type Solanum plant cultured under heat conditions being sub-optimal thereto, provides reduced yield. In some embodiments, culturing a wild-type Solanum plant under heat conditions being sub-optimal thereto, as disclosed herein, comprises reducing: number of fruit, total yield (gr), harvest index, number of inflorescences, number of fruit per inflorescence, % of parthenocarpic fruit, or any combination thereof, of the wild-type Solanum plant.

In some embodiments, a pathogen is selected from: a fungus, an oomycete, a bacterium, a virus, an arthropod, or any combination thereof.

In some embodiments, a pathogen is selected from a fungus or a bacterium.

In some embodiments, a fungus is selected from the genera: Botrytis, Alternaria, Oidium, Sclerotinia, Sclerotium rolfsii, Fusarium, Leveillula, Lasodiplodia, Penicillium, Aspergillus, Talaromyces, Macrophomina, Verticillium, Cladosporium, or any combination thereof. In some embodiments, a fungus is selected from the species: Botrytis cinerea, Botrytis elliptica, Sclerotinia sclerotiorum, Sclerotium rolsfii, Alternaria alternata, Alternaria Solani, Fusarium oxysporum, Fusarium solani, Oidium lycopersici, Oidium neolycoperisi, Leveillula taurica, Penicillium digitatum, Penicillium expansum, Aspergillus niger, Macrophomina phaseolina, Verticillium dahliae, Cladosporium fulvum, or any combination thereof. In some embodiments, a fungus is selected from: Botrytis cinerea, Oidium neolycopersici, Alternaria alternata, or any combination thereof.

In some embodiments, an oomycete is selected from the genera: Pythium, Phytopthora, or both. In some embodiments, an oomycete is selected from the species: Phythium spinosum, Phytium afanidermatum, Phytopthora infestans, Phytophthora lycopersici, or any combination thereof.

In some embodiments, a bacterium is selected from the genera: Clavibacter, Pseudomonas, Xanthomonas, Xyllela, Erwinia, Liberibacter, or any combination thereof. In some embodiments, a bacterium is selected from the species: Clavibacter michigenensis, Pseudomonas syringae, Pseudomoas corrugata, Xanthomonas euvesicatoria, or any combination thereof. In some embodiments, a bacterium comprises or consists of Xanthomonas euvesicatoria.

In some embodiments, a virus is selected from: Tobamoviruses, Mosaic viruses, Peppino Viruses, Gemini viruses, or any combination thereof. In some embodiments, a virus is selected from: TMV, ToMV, TSWV, ToBRFV, TYLCV, or any combination thereof.

In some embodiments, an arthropod is selected from: an insect, an arachnid, or both. In some embodiments, an arthropod is selected from: a fly, a mite, a leafhopper, a leafminer, or any combination thereof. In some embodiments, an arthropod is selected from: Bemicia tabaci, Tetranychus urticae, Tuta absoluta, or any combination thereof.

Types of Solanum plants as well as methods for determining a plant belongs to the Solanum genus (of the Solanaceae family), are common and would be apparent to one of ordinary skill in the art. Non-limiting example for determination of a plant belonging to the Solanum genus comprises utilization of a Flora (publication).

In some embodiments, a genetically modified Solanum plant as disclosed herein is characterized by having a genome comprising at least two paralogs of ARF8 gene. In some embodiments, a genetically modified Solanum plant as disclosed herein is characterized by having a genome comprising two or more paralogs of ARF8 gene. In some embodiments, a genetically modified Solanum plant as disclosed herein is characterized by having a genome comprising a plurality of paralogs of ARF8 gene. In some embodiments, a genetically modified plant, as disclosed herein, is or comprises a S. lycopersicum (tomato) plant. In some embodiments, a genetically modified plant, as disclosed herein, is or comprises a S. melongena (eggplant) plant. In some embodiments, a genetically modified plant, as disclosed herein, is or comprises a S. melongena (pepper) plant.

As used herein, the term “plurality” refers to any integer being equal to or greater than 2.

In some embodiments, any one of: at least two, one or more, or plurality of ARF8 gene paralogs comprise: (i) at least a first ARF8 gene paralog being ARF8a gene; and (ii) at least a second ARF8 gene paralog not being ARF8a gene paralog.

In some embodiments, any one of: at least two, one or more, or plurality of ARF8 gene paralogs comprise: (i) at least a first ARF8 gene paralog being ARF8b gene; and (ii) at least a second ARF8 gene paralog not being ARF8b gene paralog.

In some embodiments, any one of: at least two, one or more, or plurality of ARF8 gene paralogs comprise: (i) at least a first ARF8 gene paralog being ARF8a gene; and (ii) at least a second ARF8 gene paralog being ARF8b gene paralog.

In some embodiments, any one of the ARF8 gene paralogs as disclosed herein, refer to ARF8 gene paralogs of Solanum lycopersicum. In some embodiments, any one of the ARF8 gene paralogs as disclosed herein, refer to orthologs thereof in a Solanum plant being other than S. lycopersicum. In some embodiments, ARF8 gene paralogs as disclosed herein, refer to any one of ARF8a paralog of S. lycopersicum, ARF8b paralog of S. lycopersicum, both, or an ortholog(s) thereof. In some embodiments, an ortholog(s) is in/of a Solanum plant being other than S. lycopersicum.

As used herein, the terms “active”, “inactive”, “activity”, “inactivity” relates to auxin responsiveness, auxin-dependent signaling, auxin-dependent gene activation, expression, transcription, or any combination thereof.

In some embodiments, “active” or “activity” comprises increasing, promoting, enhancing, propagating, or any combination thereof, any one of auxin responsiveness, auxin-dependent signaling, auxin-dependent gene activation, expression, transcription, or any combination thereof.

In some embodiments, “inactive” or “inactivity” comprises reducing or inhibiting any one of auxin responsiveness, auxin-dependent signaling, auxin-dependent gene activation, expression, transcription, or any combination thereof.

In some embodiments, inactive comprises partially inactive, fully inactive, or both.

In some embodiments, fully inactive is inhibited, e.g., 100% inactivity, compared to a control (such as a wild-type allele). In some embodiments, fully inactive is inhibited, e.g., 0% activity, compared to a control (such as a wild-type allele). In some embodiments, partially is reduced, e.g., not more than 99% activity, compared to a control (such as a wild-type allele). In some embodiments, partially is reduced, e.g., not less than 99% inactivity, compared to a control (such as a wild-type allele).

In some embodiments, auxin responsiveness, auxin-dependent signaling, auxin-dependent gene activation, expression, transcription, or any combination thereof, comprises: auxin-dependent signal transduction or signaling, DNA binding, DNA binding in or of auxin-responsive promoter element(s) (AuxRE(s)), controlling or regulating stamen maturation, gynoecium maturation, or both, promoting, enhancing, increasing, propagating, any equivalent thereof, or any combination thereof, of jasmonic acid production, reducing or inhibiting fruit setting, inhibiting carpel development in the absence of fertilization, or any combination thereof.

In some embodiments, culturing under heat conditions comprises subjecting a genetically modified plant, as disclosed herein to: temperature ranging from 26° C. to 45° C., 28° C. to 44° C., 29° C. to 43° C., 31° C. to 45° C., or 26° C. to 42° C. Each possibility represents a separate embodiment of the invention.

In some embodiments, culturing under heat conditions comprises subjecting a genetically modified plant, as disclosed herein to a temperature of at least: 26° C., 32° C., 35° C., 38° C., 41° C., or 44° C., or any value and range therebetween. Each possibility represents a separate embodiment of the invention.

In some embodiments, culturing under heat conditions comprises subjecting a genetically modified plant as disclosed herein, to a temperature as disclosed herein throughout the day. In some embodiments, culturing under heat conditions comprises subjecting a genetically modified plant as disclosed herein, to a temperature as disclosed herein for 12-16 hours a day.

In some embodiments, culturing under heat conditions comprises subjecting a genetically modified plant as disclosed herein, to a temperature as disclosed herein throughout the night. In some embodiments, culturing under heat conditions comprises subjecting a genetically modified plant as disclosed herein, to a temperature as disclosed herein for 8-12 hours a day (i.e., during the night).

In some embodiments, culturing under cold conditions comprises subjecting a genetically modified plant, as disclosed herein to: temperature ranging from 6° C. to 19° C., 8° C. to 18° C., 9° C. to 15° C., 5° C. to 20° C., or 6° C. to 18° C. Each possibility represents a separate embodiment of the invention.

In some embodiments, culturing under cold conditions comprises subjecting a genetically modified plant, as disclosed herein to a temperature of at least: 6° C., 8° C., 10° C., 14° C., 16° C., or 18° C., or any value and range therebetween. Each possibility represents a separate embodiment of the invention.

In some embodiments, culturing under cold conditions comprises subjecting a genetically modified plant as disclosed herein, to a temperature as disclosed herein throughout the day. In some embodiments, culturing under cold conditions comprises subjecting a genetically modified plant as disclosed herein, to a temperature as disclosed herein for 12-16 hours a day.

In some embodiments, culturing under cold conditions comprises subjecting a genetically modified plant as disclosed herein, to a temperature as disclosed herein throughout the night. In some embodiments, culturing under cold conditions comprises subjecting a genetically modified plant as disclosed herein, to a temperature as disclosed herein for 8-12 hours a day (i.e., during the night).

In some embodiments, culturing as disclosed herein, is for a period ranging from 5 to 130 days, 10 to 130 days, 20 to 130 days, 20 to 100 days, 15 to 125 days, or 10 to 90 days. Each possibility represents a separate embodiment of the invention.

In some embodiments, culturing as disclosed herein, is for a period of at least: 1 day, 3 days, 5 days, 7 days, 10 days, 15 days, 20 days, 50 days, or any value and range therebetween. Each possibility represents a separate embodiment of the invention.

In some embodiments, culturing under sub-optimal conditions as disclosed herein, is for a period of at least 1-2 hours a day, 1-5 hours a day, or 1 to 8 hours a day. Each possibility represents a separate embodiment of the invention.

In some embodiments, culturing under heat stress, cold stress, or both, as disclosed herein, is for a period of at least 1-2 hours a day, 1-5 hours a day, or 1 to 8 hours a day. Each possibility represents a separate embodiment of the invention.

In some embodiments, the at least one allele of any one of: the ARF8a gene, the ARF8b gene, or both, being inactive is: knocked out, mutated, or knocked down, such that an mRNA transcribed therefrom, a protein product translated therefrom, or both, is inactive or absent, in a genetically modified plant, as disclosed herein. In some embodiments, the at least one allele of any one of: the ARF8a gene, the ARF8b gene, or both, being fully inactive is: knocked out, mutated, or knocked down, such that an mRNA transcribed therefrom, a protein product translated therefrom, or both, is inactive or absent, in a genetically modified plant, as disclosed herein. In some embodiments, two alleles of any one of the inactive: ARF8a gene, ARF8b gene, or both, are: knocked out, mutated, or knocked down, such that an mRNA transcribed therefrom, a protein product translated therefrom, or both, is inactive or absent, in the genetically modified plant.

In some embodiments, a genetically modified or gene edited plant as disclosed herein, comprising inactive allele of the ARF8 gene, has a reduced amount of mRNA transcribed from the allele of the ARF8 gene or is devoid therefrom, has a reduced amount of a protein product encoded from the allele of the ARF8 gene or is devoid therefrom, has reduced protein activity of the protein product encoded from the allele of the ARF8 gene or is devoid therefrom, or any combination thereof.

In some embodiments, a genetically modified or gene edited plant, as disclosed herein, is characterized by inhibited or reduced auxin responsiveness or signaling. In some embodiments, a genetically modified or gene edited plant, as disclosed herein, is characterized by inhibited or reduced auxin responsiveness or signaling via a protein product of ARF8a gene, ARF8b gene, or both. In some embodiments, a genetically modified or gene edited plant, as disclosed herein, comprises at least one cell having: a reduced amount of mRNA transcribed from the allele of ARF8a gene, ARF8b, or both, or is devoid therefrom, a reduced amount of a protein product encoded from the allele of ARF8a gene, ARF8b gene, or both, or is devoid therefrom, has a protein product encoded from the allele of ARF8a gene, ARF8b gene, or both having reduced activity, e.g., auxin-dependent signaling as disclosed herein, or is devoid thereof, or any combination thereof.

In some embodiments, reduced protein activity relates to a protein having reduced or no capability to induce or promote an auxin responsiveness, auxin-dependent signaling, auxin-dependent gene activation, expression, transcription, or any combination thereof, as disclosed herein.

In some embodiments “partially” is compared to a control, as disclosed herein, such as, but not limited to a wild-type allele of a plant or a cell thereof. In some embodiments, partially comprises 99% at most, 95% at most, 90% at most, 80% at most, 70% at most, 60% at most, 50% at most, 40% at most, 30% at most, 20% at most, 10% at most, 5% at most, or 1% at most, compared to a control, or any value and range therebetween. Each possibility represents a separate embodiment of the invention. In some embodiments, partially comprises 1-99%, 1-95%, 1-90%, 1-80%, 1-70%, 1-60%, 1-50%, 1-40%, 1-30%, 1-20%, 1-10%, or 1-5%, compared to a control. Each possibility represents a separate embodiment of the invention. In some embodiments, a control comprises a fully active allele, such as, but not limited to, a wild-type allele of ARF8 gene of a plant (e.g., wild-type Solanum plant). In some embodiments, a fully active allele of ARF8 gene encodes an active and/or functional protein product.

In some embodiments, an inactive allele as disclosed herein is not transcribed. In some embodiments, an inactive allele as disclosed herein is partially transcribed, e.g., to reduced amount, rate, or both, compared to control. In some embodiments, an inactive allele as disclosed herein is transcribed (e.g., giving rise to an mRNA), and is not translated to a protein. In some embodiments, a transcript transcribed from an inactive allele as disclosed herein, is degraded, and is not translated to a protein. In some embodiments, degradation is promoted or induced by an inhibitory polynucleotide or an oligonucleotide, e.g., via RNA interference (RNAi). In some embodiments, a transcript transcribed from an inactive allele as disclosed herein, is knocked down. In some embodiments, an inhibitory polynucleotide or oligonucleotide, e.g., “an RNAi agent”, inducing or promoting the degradation of the transcript of the ARF8 gene is incorporated into the genome of the genetically modified plant disclosed herein, and is transcribed therefrom (e.g., endogenous knock down). In some embodiments, an inhibitory polynucleotide or oligonucleotide, e.g., “an RNAi agent” inducing or promoting the degradation of the transcript of the ARF8 gene is exogenously applied to the genetically modified plant disclosed herein (e.g., exogenous knock down). In some embodiments, a genetically modified plant as disclosed herein, comprises a genome further comprising at least one nucleic acid sequence configured to knocking down the levels, stability, or both, of a transcript of the ARF8 gene. In some embodiments, configured to comprises having a sequence complementarity level sufficient to induce or activate an RNA-induced silencing complex (RISC) response/activity. In some embodiments, the genome of the genetically modified plant disclosed herein further comprises a nucleic acid sequence transcribed into an inhibitory polynucleotide or oligonucleotide targeting a transcript transcribed from the ARF8 gene or allele, as disclosed herein. In some embodiments, an inactive allele as disclosed herein is fully or partially knocked out from the genome of a genetically modified plant as disclosed herein. In some embodiments, fully knocked out is to be understood such that the full protein encoding sequence of an ARF8 gene is absent from the genome of the genetically modified plant as disclosed herein. In some embodiments, partially knocked out is to be understood such that the allele of the ARF8 gene is incomplete compared to the wild-type allele of ARF8 gene, such that it is being devoid of a nucleic acid sequence encoding a fragment of the protein product of the gene. In some embodiments, partially knocked out it to be understood such that the allele of the ARF8 gene comprises a premature stop codon (e.g., a nonsense mutation). In some embodiments, partially knocked out it to be understood such that the allele of the ARF8 gene is encoding a deleterious or a truncated protein. In some embodiments, a partially or fully knocked out allele of the ARF8 gene does not encode a functional and/or active protein product. In some embodiments, a transcript transcribed from the allele of the ARF8 gene is translation-inhibited. In some embodiments, translation inhibition or translation-inhibited is by being bound to a microRNA (miRNA) or a mimetic thereof. In some embodiments, the miRNA or a mimetic thereof is miRNA 167 (GenBank Accession No. NR_107987). In some embodiments, a genetically modified plant as disclosed herein is characterized by over-expression of a gene encoding miRNA 167. In some embodiments, a genetically modified plant as disclosed herein is characterized by having increased levels of miRNA 167, compared to a control plant, e.g., wild-type plant.

### Genetically Modified Solanum Plant

According to another aspect, there is provided a genetically modified Solanum plant comprising: at least one inactive allele of ARF8a gene, at least one inactive allele of ARF8b gene, or both, wherein the genetically modified Solanum plant comprises a genome being devoid of any one of: (i) one or both alleles of ARF8a gene comprising the nucleic acid sequence: ATGAAGCTTTCAACATGGAATGGGTCCAGCAAGCTCATGA (SEQ ID NO: 29), ATGAAGCTTTCCATCAGGAATGGGTCCAGCAAGCTCATGA (SEQ ID NO: 30), or both; (ii) one or both alleles of ARF8b gene comprising the nucleic acid sequence: ATGAAGCTTTCAACATCAGAGAATGGGTCAGCAGGCTCATGA (SEQ ID NO: 31), ATGAAGCTTTCTCAGGAATGGGTCAGCAGGCTCATGAAGGAGGAGAGAAAAAGTGT TTGA (SEQ ID NO: 32), or both; or (iii) both (i) and (ii).

In some embodiments, “devoid” is to meant that a nucleic acid being a DNA sequence or an RNA sequence comprising any one of SEQ ID Nos: 29-31 are absent in a cell of the genetically modified Solanum plant of the invention. In some embodiments, a DNA sequence comprising any one of SEQ ID Nos: 29-31 is absent from the genome of the genetically modified Solanum plant of the invention, or of a cell thereof. In some embodiments, the genome of the genetically modified Solanum plant of the invention, or of a cell thereof, does not include a DNA sequence comprising any one of SEQ ID Nos: 29-31.

In some embodiments, an RNA sequence comprising any one of SEQ ID Nos: 29-31 is absent from the transcriptome of the genetically modified Solanum plant of the invention, or of a cell thereof. In some embodiments, the transcriptome of the genetically modified Solanum plant of the invention, or of a cell thereof, does not include an RNA sequence comprising any one of SEQ ID Nos: 29-31.

In some embodiments, the genetically modified Solanum plant comprises at least one inactive allele of the ARF8a gene comprising the nucleic acid sequence selected from:

(SEQ ID NO: 25)
AATTACCCCGAACTTGCCACCACAGCTGTCAACTCCACAATGTCAC;
or
(SEQ ID NO: 26)
AATTACCCCGAACTTGCCACCACAGTTTGATCTGTCAACTCCACAATGT
CAC.

In some embodiments, the genetically modified Solanum plant comprises at least one inactive allele of the ARF8b gene comprising the nucleic acid sequence:

(SEQ ID NO: 27)
CTCTGGATCTGTCAACTCCACA.

In some embodiments, the genetically modified Solanum plant comprises: (i) at least one inactive allele of the ARF8a gene comprising the nucleic acid sequence set forth in SEQ ID NO: 25 and at least one inactive allele of the ARF8b gene comprising the nucleic acid sequence set forth in SEQ ID NO: 27; or (ii) at least one inactive allele of the ARF8a gene comprising the nucleic acid sequence set forth in SEQ ID NO: 26 and at least one inactive allele of the ARF8b gene comprising the nucleic acid sequence set forth in SEQ ID NO: 27.

In some embodiments, the at least one inactive allele of ARF8a gene, ARF8b gene, or both, is fully inactive.

In some embodiments, both alleles of ARF8a gene, ARF8b gene, or both, are fully inactive.

In some embodiments, the genetically modified Solanum plant comprises: (i) two fully inactive alleles of the ARF8a gene comprising the nucleic acid sequence set forth in SEQ ID NO: 25 and two fully inactive alleles of the ARF8b gene comprising the nucleic acid sequence set forth in SEQ ID NO: 27; or (ii) two fully inactive alleles of the ARF8a gene comprising the nucleic acid sequence set forth in SEQ ID NO: 25 and two fully inactive alleles of the ARF8b gene comprising the nucleic acid sequence set forth in SEQ ID NO: 27.

In some embodiments, a plant part comprises a cell, a tissue, a fragment, or any combination thereof, of a plant, e.g., a Solanum plant.

In some embodiments, a plant part comprises: a leaf, a stem, a root, a floral organ or structure, pollen, a seed, a seed part such as an embryo, endosperm, scutellum or seed coat, a plant tissue such as, for example, vascular tissue, a cell, or any combination thereof.

In some embodiments, a genetically modified plant of the invention comprises a premature stop codon in at least one allele of the ARF8a gene. In some embodiments, a genetically modified plant of the invention comprises a premature stop codon in both alleles of the ARF8a gene. In some embodiments, a genetically modified plant of the invention comprises a premature stop codon in at least one allele of the ARF8b gene. In some embodiments, a genetically modified plant of the invention comprises a premature stop codon in both alleles of the ARF8b gene. In some embodiments, a genetically modified plant of the invention comprises a premature stop codon in at least one allele of the ARF8a gene and a premature stop codon in at least one allele of the ARF8b gene. In some embodiments, a genetically modified plant of the invention comprises premature stop codons in both alleles of the ARF8a gene and premature stop codons in both alleles of the ARF8a gene.

In some embodiments, a wild-type allele of the ARF8a gene comprises the nucleic acid sequence:

(SEQ ID NO: 23)
AATTACCCGAACTTGCCACCACAGTTGATCTGTCAACTCCACAATGTCA
CA.

In some embodiments, a wild-type allele of the ARF8b gene comprises the nucleic acid sequence:

(SEQ ID NO: 24)
CTCTGGTGTGCCTACCAACTGTCGGAAGTCGAGTTGTTTACTTTCCTCA
GGGTCATAGTGAACAGGTCTTTCTCCTTTTCATTCTTTATCACACAAAC
TGATACAATCTATTATTTTAGTTATAAATCACTGATGTTCCCTTACTGG
AATGGAGTGACAGGTAGCTGCAACAACTAATAAAGAAGTTGATGCACAT
ATACCCAATTACCCAAATTTGTCGCCGCAGTTGATCTGTCAACTCCA.

In some embodiments, a genetically modified plant of the invention comprises a genome comprising the nucleic acid sequence set forth in SEQ ID NO: 23 with a deletion of at least one nucleotide of SEQ ID NO: 23. In some embodiments, the genetically modified plant of the invention comprises a genome comprising the nucleic acid sequence set forth in SEQ ID NO: 23 with a deletion of 5 consecutive nucleotides in positions 26-30 of SEQ ID NO: 23. In some embodiments, the genetically modified plant of the invention comprises a genome comprising the nucleic acid sequence set forth in SEQ ID NO: 23 with a deletion of the nucleic acid sequence: TTGAT (SEQ ID NO: 28) from SEQ ID NO: 23.

In some embodiments, a genetically modified plant of the invention, comprises one or more alleles of the ARF8a gene comprising the nucleic acid sequence:

(SEQ ID NO: 25)
AATTACCCGAACTTGCCACCACAGCTGTCAACTCCACAATGTCACA.

In some embodiments, a genetically modified plant utilized according to the claimed invention, comprises one or more alleles of the ARF8a gene comprising the nucleic acid sequence:

(SEQ ID NO: 26)
AATTACCCGAACTTGCCACCACAGTTTGATCTGTCAACTCCACAATGTC
ACA.

In some embodiments, a genetically modified plant of the invention, comprises one or more alleles of the ARF8b gene comprising the nucleic acid sequence:

(SEQ ID NO: 27)
CTCTGGATCTGTCAACTCCACA.

In some embodiments, a genetically modified plant of the invention comprises one or more alleles of the ARF8a gene comprising the nucleic acid sequence set forth in any one of SEQ ID Nos: 25-26, and one or more alleles of the ARF8b gene comprising the nucleic acid sequence set forth in SEQ ID NO: 27.

In some embodiments, a genetically modified plant of the invention is produced using single guide RNA (sgRNA) and CRISPR-Cas system, as further disclosed herein.

In some embodiments, sgRNA used in the production or manufacture of a genetically modified plant as disclosed herein, comprises the nucleic acid sequence: CAGTTGATCTGTCAACTCCA (SEQ ID NO: 21). In some embodiments, sgRNA used in the production or manufacture of a genetically modified plant as disclosed herein, comprises the nucleic acid sequence: TGGAGTTGACAGATCAACTG (SEQ ID NO: 22). In some embodiments, sgRNA used in the production or manufacture of a genetically modified plant as disclosed herein, comprises the nucleic acid sequence: ACAGTTGGTAGGCACACCAG (SEQ ID NO: 33)

In some embodiments, the method further comprises a step preceding the culturing step, comprising producing, or manufacturing the genetically modified plant as disclosed herein.

In some embodiments, the producing, or manufacturing comprises contacting a wild-type Solanum plant, a cell thereof, or a part thereof with CRISPR-Cas system comprising at least one sgRNA comprises a nucleic acid sequence set forth in SEQ ID Nos: 21-22, and 33.

In some embodiments, a genetically modified plant as disclosed herein, comprising an inactive allele of the ARF8a gene, is produced by contacting a wild-type Solanum plant, a cell thereof, or a part thereof with CRISPR-Cas system comprising a sgRNA comprising nucleic acid sequence set forth in SEQ ID NO: 21.

In some embodiments, a genetically modified plant as disclosed herein, comprising an inactive allele of the ARF8b gene, is produced by contacting a wild-type Solanum plant, a cell thereof, or a part thereof with CRISPR-Cas system comprising a sgRNA comprising nucleic acid sequence set forth in any one of SEQ ID Nos: 22 and 33.

In some embodiments, a genetically modified plant as disclosed herein, comprising an inactive allele of the ARF8a gene, and an inactive allele of the ARF8b gene is produced by contacting a wild-type Solanum plant, a cell thereof, or a part thereof with CRISPR-Cas system comprising a plurality of sgRNAs comprising a nucleic acid sequence set forth in SEQ ID NO: 21 and a nucleic acid sequence set forth in any one of SEQ ID Nos: 22, 33, and both.

In some embodiments, a genetically modified Solanum plant of the invention is characterized by: (i) increased yield; (ii) increased resistance to a pathogen; (iii) earlier fruit setting; or (iv) any combination of (i) to (iii), compared to a wild-type variant of said Solanum plant.

As used herein, the terms “genetically modified plant of the invention”, “genetically modified Solanum plant of the invention”, and “genetically modified Solanum plant of the invention” are interchangeable.

Methods for gene or genetic editing/transgenesis/genetic modifications, are common and would be apparent to one of ordinary skill in the art of molecular biology. Non-limiting example for means and methods of gene editing, includes, but is not limited to the utilization of a CRISR-Cas system, such as exemplified herein below.

Methods for determining the presence of a sequence, in general, and specifically such that includes a premature stop codon (or a nonsense mutation), are common and would be apparent to one of ordinary skill in the art. Non-limiting examples for such methods, include, but are not limited to, PCR, sequencing, such as, but not limited to Sanger sequencing, next generation sequencing, restriction fragment length polymorphism (RFLP), among other.

In some embodiments, increasing is compared to a control plant, as disclosed herein, or a cell thereof.

In some embodiments, a control plant (or a cell thereof) is characterized by having a genome comprising two wild-type alleles of: ARF8a gene, ARF8b gene, or both, such that an mRNA transcribed therefrom, a protein product translated therefrom, or both, is active or present, in the control plant. In some embodiments, a control plant is characterized by having a genome comprising two alleles of: ARF8a gene, ARF8b gene, or both, being active, such that an mRNA transcribed therefrom, a protein product translated therefrom, or both, is active or present, in the control plant.

In some embodiments, the inhibitory polynucleotide or oligonucleotide comprises an antisense polynucleotide or oligonucleotide.

As used herein, an “antisense polynucleotide or oligonucleotide” refers to a nucleic acid sequence that is reversed and complementary to a DNA or RNA sequence of the allele of the ARF8 gene or a transcript thereof, respectively.

As referred to herein, a “reversed and complementary nucleic acid sequence” is a nucleic acid sequence capable of hybridizing with another nucleic acid sequence comprised of complementary nucleotide bases. By “hybridize” is meant pair to form a double-stranded molecule between complementary nucleotide bases (e.g., adenine (A) forms a base pair with thymine (T) (or uracil (U) in the case of RNA), and guanine (G) forms a base pair with cytosine (C)) under suitable conditions of stringency. (See, e.g., Wahl, G. M. and S. L. Berger (1987) Methods Enzymol. 152:399; Kimmel, A. R. (1987) Methods Enzymol. 152:507). For the purposes of the present methods, the inhibitory nucleic acid need not be complementary to the entire sequence, only enough of it to provide specific inhibition; for example, in some embodiments the sequence is 100% complementary to at least nucleotides (nts) 2-7 or 2-8 at the 5′ end of the microRNA itself (e.g., the ‘seed sequence’), e.g., nts 2-7 or 20.

In some embodiments of the inhibitory polynucleotide or oligonucleotide has one or more chemical modifications to the backbone or side chains. In some embodiments, the inhibitory nucleic acid has at least one locked nucleotide, and/or has a phosphorothioate backbone.

Non-limiting examples of inhibitory polynucleotide or oligonucleotide useful according to the herein disclosed invention include, but are not limited to: antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof.

In some embodiments, the inhibitory polynucleotide or oligonucleotide is an RNA interfering molecule (RNAi) agent. In some embodiments, the RNAi agent is or comprises double stranded RNA (dsRNA).

As used herein “an interfering RNA” refers to any double stranded or single stranded RNA sequence, capable either directly or indirectly (i.e., upon conversion) of inhibiting or down regulating gene expression by mediating RNA interference. Interfering RNA include but are not limited to small interfering RNA (“siRNA”) and small hairpin RNA (“shRNA”). “RNA interference” refers to the selective degradation of a sequence-compatible messenger RNA transcript.

The term “gene edited plant” refers to a plant comprising at least one cell comprising at least one gene edited by man. The gene editing includes deletion, insertion, silencing, or repression, such as of the “native genome” of the cell. Methods for creating a gene edited plant include techniques such as zinc-finger nucleases (ZFN), transcription activator-like effector nucleases (TALEN), and clustered regularly interspersed short palindromic repeats (CRISPR)/Cas systems.

The term “genetically modified plant” refers to a plant comprising at least one cell genetically modified by man. The genetic modification includes modification of an endogenous gene(s), for example by introducing mutation(s) deletions, insertions, transposable element(s) and the like into an endogenous polynucleotide or gene of interest. Additionally, or alternatively, the genetic modification includes transforming the plant cell with heterologous polynucleotide. A “genetically modified plant” and a “corresponding unmodified plant” as used herein refer to a plant comprising at least one genetically modified cell and to a plant of the same type lacking the modification, respectively. A “genetically modified plant” encompasses a plant or a cell thereof, comprising a genome being altered compared to the genome of a wild-type plant or a cell thereof. In some embodiments, altered comprises: having an insertion, deletion, inversion, mutation, or any combination thereof. In some embodiments, a mutation is a nonsense mutation or a non-synonymous mutation. In some embodiments, genetically modified comprises a transgene. In some embodiments, genetically modified comprises genetically edited. The terms: (i) “transgene” or “transgenic”; (ii) “genetically modified”; and (iii) “genetically edited” are used herein interchangeably.

One of ordinary skill in the art would appreciate that a genetically modified plant may encompass a plant comprising at least one cell genetically modified by man. In some embodiments, the genetic modification includes modification of an endogenous gene(s), for example by introducing mutation(s) deletions, insertions, transposable element(s) and the like into an endogenous polynucleotide or gene of interest. Additionally, or alternatively, in some embodiments, the genetic modification includes transforming at least one plant cell with a heterologous polynucleotide or multiple heterologous polynucleotides. The skilled artisan would appreciate that a genetically modified plant comprising transforming at least one plant cell with a heterologous polynucleotide or multiple heterologous polynucleotides may in certain embodiments be termed a “transgenic plant”.

A skilled artisan would appreciate that a comparison of a “genetically modified plant” to a “corresponding unmodified plant” as used herein encompasses comparing a plant comprising at least one genetically modified cell and to a plant of the same type lacking the modification.

The skilled artisan would appreciate that the term “transgenic” when used in reference to a plant as disclosed herein encompasses a plant that contains at least one heterologous transcribable polynucleotide in one or more of its cells. The term “transgenic material” encompasses broadly a plant or a part thereof, including at least one cell, multiple cells or tissues that contain at least one heterologous polynucleotide in at least one of cell. Thus, comparison of a “transgenic plant” and a “corresponding non transgenic plant”, or of a “genetically modified plant comprising at least one cell having altered expression, wherein the plant comprising at least one cell comprising a heterologous transcribable polynucleotide” and a “corresponding unmodified plant” encompasses comparison of the “transgenic plant” or “genetically modified plant” to a plant of the same type lacking the heterologous transcribable polynucleotide. A skilled artisan would appreciate that, in some embodiments, a “transcribable polynucleotide” comprises a polynucleotide that can be transcribed into an RNA molecule by an RNA polymerase.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

Transformation of a cell may be stable or transient. The term “transient transformation” or “transiently transformed” refers to the introduction of one or more exogenous polynucleotides into a cell in the absence of integration of the exogenous polynucleotide into the host cell's genome. In contrast, the term “stable transformation” or “stably transformed” refers to the introduction and integration of one or more exogenous polynucleotides into the genome of a cell. The term “stable transformant” refers to a cell which has stably integrated one or more exogenous polynucleotides into the genomic or organellar DNA. It is to be understood that an organism or its cell transformed with the nucleic acids, constructs and/or vectors of the present invention can be transiently as well as stably transformed.

The skilled artisan would appreciate that the term “construct” may encompass an artificially assembled or isolated nucleic acid molecule which includes the polynucleotide of interest. In general, a construct may include the polynucleotide or polynucleotides of interest, a marker gene which in some cases can also be a gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

The skilled artisan would appreciate that the term “expression” may encompass the production of a functional or nonfunctional end-product e.g., an mRNA or a protein.

In some embodiments, genetically edited plant as disclosed herein, is obtained by gene editing methodology. In some embodiments, gene editing of a genome is achieved by utilizing at least one programmable engineered nuclease (PEN).

In some embodiments, a PEN is a clustered regularly interspaced short palindromic repeat (CRISPR) type II system.

In some embodiments, CRISPR type II system comprises CRISPR-associated protein 9 (Cas9).

The term “programmable engineered nucleases (PEN)” as used herein, refers to synthetic enzymes that cut specific DNA sequences, derived from natural occurring nucleases involved in DNA repair of double strand DNA lesions and enabling direct genome editing.

In some embodiments, PEN used by the methods of the invention may be any one of a clustered regularly interspaced short palindromic repeat (CRISPR) Class 2 or Class 1 system.

The clustered regularly interspaced short palindromic repeats (CRISPR) Type II system is a bacterial immune system that has been modified for genome engineering. It should be appreciated however that other genome engineering approaches, like zinc finger nucleases (ZFNs) or transcription-activator-like effector nucleases (TALENs) that relay upon the use of customizable DNA-binding protein nucleases that require design and generation of specific nuclease-pair for every genomic target may be also applicable herein.

CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI.

As used herein, “CRISPR arrays” also known as SPIDRs (Spacer Interspersed Direct Repeats) constitute a family of recently described DNA loci that are usually specific to a particular bacterial species. The CRISPR array is a distinct class of interspersed short sequence repeats (SSRs) that were first recognized in E. coli. In subsequent years, similar CRISPR arrays were found in Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly and of the CRISPR systems disclosed herein. The CRISPR-Cas system has evolved in prokaryotes to protect against phage attack and undesired plasmid replication by targeting foreign DNA or RNA. The CRISPR-Cas system, targets DNA molecules based on short homologous DNA sequences, called spacers that exist between repeats. These spacers guide CRISPR-associated (Cas) proteins to matching (and/or complementary) sequences within the foreign DNA, called proto-spacers, which are subsequently cleaved. The spacers can be rationally designed to target any DNA sequence. Moreover, this recognition element may be designed separately to recognize and target any desired target. With respect to CRISPR systems, as will be recognized by those skilled in the art, the structure of a naturally occurring CRISPR locus includes a number of short repeating sequences generally referred to as “repeats”. The repeats occur in clusters and are usually regularly spaced by unique intervening sequences referred to as “spacers.” Typically, CRISPR repeats vary from about 24 to 47 base pair (bp) in length and are partially palindromic. The spacers are located between two repeats and typically each spacer has unique sequences that are from about 20 or less to 72 or more bp in length. In some embodiments the CRISPR spacers used in the sequence encoding at least one sgRNA of the methods and kits of the invention comprise between 10 to 75 nucleotides (nt) each. In some embodiments, the sgRNA comprises at least: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or any vale and range therebetween. Each possibility represents a separate embodiment of the invention. In some embodiments, the sgRNA comprises 70 to 150 nt. In some specific embodiments the spacers comprise 20 to 35 nucleotides.

In addition to at least one repeat and at least one spacer, a CRISPR locus also includes a leader sequence and optionally, a sequence encoding at least one tracrRNA. The leader sequence typically is an AT-rich sequence of up to 550 bp directly adjoining the 5′ end of the first repeat.

In some embodiments, the PEN used by the methods of the invention may be a CRISPR Class 2 system. In yet some further particular embodiments, such class 2 system may be a CRISPR type II system.

More specifically, three major types of CRISPR-Cas system are delineated: Type I, Type II and Type III.

The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas 1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage.

Type II systems cleave the pre-crRNA through an unusual mechanism that involves duplex formation between a tracrRNA and part of the repeat in the pre-crRNA; the first cleavage in the pre-crRNA processing pathway subsequently occurs in this repeat region. Still further, it should be noted that type II system comprise at least one of Cas9, Cas1, Cas2 csn2, and Cas4 genes. It should be appreciated that any type II CRISPR-Cas systems may be applicable in the present invention, specifically, any one of type II-A or B.

In some embodiments, the at least one Cas gene used in the methods and kits of the invention may be at least one Cas gene of type II CRISPR system (either type II-A or type II-B). In some embodiments, at least one Cas gene of type II CRISPR system used by the methods and kits of the invention is the Cas9 gene. It should be appreciated that such system may further comprise at least one of Cas1, Cas2, csn2 and Cas4 genes.

In some embodiments, a Cas protein consists or comprise a Cas9 protein.

Double-stranded DNA (dsDNA) cleavage by Cas9 is a hallmark of “type II CRISPR-Gas” immune systems. The CRISPR-associated protein Cas9 is an RNA-guided DNA endonuclease that uses RNA:DNA complementarity to identify target sites for sequence-specific double stranded DNA (dsDNA) cleavage, creating the double strand brakes (DSBs) required for the HDR that results in the integration of the reporter gene into the specific target sequence, for example, a specific target within the avian gender chromosome Z. The targeted DNA sequences are specified by the CRISPR array, which is a series of about 30 to 40 bp spacers separated by short palindromic repeats. The array is transcribed as a pre-crRNA and is processed into shorter crRNAs that associate with the Cas protein complex to target complementary DNA sequences known as proto-spacers. These proto-spacer targets must also have an additional neighboring sequence known as a proto-spacer adjacent motif (PAM) that is required for target recognition. After binding, a Cas protein complex serves as a DNA endonuclease to cut both strands at the target and subsequent DNA degradation occurs via exonuclease activity.

CRISPR type II system as used herein requires the inclusion of two essential components: a “guide” RNA (gRNA) and a non-specific CRISPR-associated endonuclease (Cas9). The sgRNA is a short synthetic RNA composed of a “scaffold” sequence necessary for Cas9-binding and about 20 nucleotide long “spacer” or “targeting” sequence which defines the genomic target to be modified. Thus, one can change the genomic target of Cas9 by simply changing the targeting sequence present in the sgRNA. Guide RNA (gRNA), as used herein refers to a synthetic fusion of the endogenous bacterial crRNA and tracrRNA, providing both targeting specificity and scaffolding/binding ability for Cas9 nuclease. Also referred to as “single guide RNA” or “sgRNA”. CRISPR was originally employed to “knock-out” target genes in various cell types and organisms, but modifications to the Cas9 enzyme have extended the application of CRISPR to “knock-in” target genes, selectively activate or repress target genes, purify specific regions of DNA, and even image DNA in live cells using fluorescence microscopy. Furthermore, the ease of generating sgRNAs makes CRISPR one of the most scalable genome editing technologies and has been recently utilized for genome-wide screens.

As used herein, the terms “genetically modified plant”, “genomically modified plant”, “transgenic plant”, “genetically edited plant”, “genomically edited plant” are interchangeable, and all refer to a plant, or a cell thereof, having an altered genome compared to a wild-type or genetic reference genome.

As used herein, the term “wild-type” is to be understood as meaning a plant which served as a starting material for the preparation of the genetically modified plant according to the invention, apart from the genetic modification introduced and resulting in a stable integration and/or expression of at least partially inactive allele encoding an ARF8 gene paralog, corresponds to that of a genetically modified plant as disclosed herein.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

### General Definitions

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8^{th} Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Plant Material and Growth Conditions (Tomato)

Tomato (Solanum lycopersicum cv M82 (LA3475)) plants were used throughout the study. Seeds were germinated and seedlings grown in a growth room or a growth chamber for 2-4 weeks. The seedlings were then transferred to a greenhouse with a natural daylight and temperature or controlled temperature in the hot and cold experiments. For field trails, the seedlings were grown in a commercial nursery and planted in the field 30 days after seeding.

The following plant materials were described before: Slarf5/Slmp, Slarf7, Slarfl9a, and Slarf19b (Israeli et al., 2019; Current Biology), Sliaa9/e (Berger et al., 2009). The Slarf8a and Slarf8b mutants were generated during this study as described below.

### Tomato Transformation and Tissue Culture

M82 seeds were used to generate transgenic tomato plants according to (McCormick, 1991) and as described in detail in (Israeli et al., 2019). Seeds were sterilized and germinated on Nitsch medium for 7-10 days, until seedlings formed cotyledons. Cotyledons were dissected and incubated over 1-2 nights. The cotyledons were then sub-cultured with 0.35-0.4 O.D. diluted agrobacterium GV3101 containing the transformation construct. The cotyledons were incubated for additional 48 hours and then moved to J1 culture media for 1-2 weeks. Appearing calli or shoots were transferred to J2 culture media for further shoot organogenesis. The culture media was replaced every two weeks until small plants formed. Plants were removed from the cotyledons and transferred into J3 culture media for further growth. After establishing a vital meristem, plants were transformed to a rooting medium, and following rooting plants were transplanted to soil for further analysis and crosses.

### Scanning Electron Microscope (SEM)

For scanning electron microscopy (SEM), tissues were fixed in 30% ethanol under vacuum for 10 min, followed by dehydration in an increasing ethanol series up to 100% ethanol. Fixed tissues were critical-point dried, mounted on a copper plate and coated with gold. Samples were viewed using a JEOL JSM-IT-100 LV microscope. The images were taken with an accelerating voltage of 10-20 kV at high vacuum (HV) mode and secondary electron image (SEI). Images were adjusted uniformly using Adobe Photoshop CS6.

### Pollen Viability Assays

Flower buds were collected from plants grown under either normal (28° C./22° C., day/night) or heat (34° C./28° C., day/night) conditions, two days after anthesis. Buds were dissected to release pollen grains, which were then fixed on a microscope slide using 10 μl of Alexander staining solution with minor adaptations as described by (Peterson et al. 2010). Viable pollen grains-stained magenta-red and non-viable grains stained blue-green. At least six images (×10 objective) were taken from each slide under a light microscope (DM500, Leica), equipped with a digital camera (ICC50 W, Leica). Pollen grains were counted using the multi-point tool from ImageJ software (Schneider et al., 2012).

### Heat and Cold Experiments

Plants were grown in pots in a growth chamber under normal temperature for 3-4 weeks, before the first flowers/inflorescence were fully developed, and then transferred to a greenhouse with controlled conditions. For heat-stress experiments, plant were grown under 34° C. Day/28° C. night. For cold stress, plants were grown under 16° C. day/10° C. night. Plants were kept under these conditions until harvest, which took place after 120 days in the heat and 150 days in the cold, when the wild-type plants ceased making fruits and were dying. For the outdoor filed experiment, plants were planted in March, and experienced several heat waves on the warmer days of the Israeli summer during the time of flowering and fruit production. Under these conditions, fruit production was severely affected in wild-type plants. The plants were harvested during August. In the second field experiment, 4-weeks old seedlings were transplanted to a brown-red degrading sandy loam soil under a high ceiled insect-proof net-house (50-mesh) in a randomized block design. The plots were drip-irrigated up to a field capacity and covered with a black mat to control weeds. Plants were grown during June-August 2021, and experienced multiple episodes of extreme heat along all growth stages. Under these conditions, fruit production was severely affected in wild-type plants. Plants were harvested after 80 days, when the first fruits began to decompose. For yield-related trait measurements, fruits were harvested and weighed individually from each plant. The number of fruit bearing inflorescence and the number of fruits in each inflorescence were recorded. Red and green fruits were counted separately, where fruits at the breaker stage were counted in the red category. Yield index was calculated as the ratio of total fruit weight to plant vegetative weight.

### RNA Extraction and RNAseq Analysis (Tomato)

RNA was extracted using the Plant/Fungi Total RNA Purification Kit (Norgen Biotek, Thorold, ON, Canada) according to the manufacturer's instructions including DNase treatment. To compare gene expression between wild-type, Slarf8a Slarf8b and Slarfl9a Slarfl9b using RNAseq, gynoecia were collected from flowers five days before anthesis, and total RNA was extracted. Two biological replicates were used. Sequencing libraries were prepared according to the Illumina TruSeq RNA protocol and sequenced on an Illumina HiSeq2000 platform.

### Accession Numbers

Sequence data used in this study can be found in the Sol Genomic Network under the following accession numbers: SlARF5/SlMP—Solyc04g081240; S1ARF6A —Solyc00g196060/Solyc12g006340; SlARF7—Solyc07g042260; S1ARF8A—Solyc03g031970; S1ARF8B—Solyc02g037530; SIARF19A—Solyc07g016180; S1ARF19B—Solyc05g047460; ENTIRE/S1IAA9—Solyc04g076850; Pistil-specific extensin-like protein—Solyc02g078100; SlGA20ox1—Solyc03g006880; SlCKX2—Solyc10g017990; SlIAA16—Solyc01g097290; MADS-box transcription factor—Solyc01g087990; SIMADS2—Solyc01g092950; AtARF6—At1g30330; AtARF7—At5g20730; AtARF8—At5g37020. STK—At4g09960.

TABLE 1
Oligonucleotides used in the current study

| | | | SEQ | |
|---|---|---|---|---|
| | | | ID | Accession |
| Name | Sequence | Use | NO | No. |
| RT- | TGACATCGAATGGAAATTCAG | qRT | 1 | Solyc03g031970 |
| S1ARF8A-F | | | | |
| RT- | GTCTCTTAGCACTAACAAACAC | qRT | 2 | |
| S1ARF8A-R | | | | |
| RT- | GTCAGTCCGTGATCATAGAG | qRT | 3 | Solyc02g037530 |
| S1ARF8B-F | | | | |
| RT- | GGAATCCAAGCTACAATTTCC | qRT | 4 | |
| S1ARF8B-R | | | | |
| RT- | TGCTGCTGTTTTTGTGGACAT | qRT | 5 | Solyc10g017990 |
| CKX2-F | | | | |
| RT- | TTCTGGTATCGTGGCTGACA | qRT | 6 | |
| CKX2-R | | | | |
| RT- | AGATTGTGTTGGTGGACTTCAA | qRT | 7 | Solyc03g006880 |
| GA20ox- | | | | |
| 1-F | | | | |
| RT- | TAGCGCCATAAATGTGTCG | qRT | 8 | |
| GA20ox- | | | | |
| 1-R | | | | |
| RT- | CCGGGGAGCTTTCTGTTCTT | qRT | 9 | Solyc01g087990 |
| MADS-F | | | | |
| RT- | GGCAGAATTATCTGTCGAGGCTA | qRT | 10 | |
| MADS-R | | | | |
| RT- | AATCTGATGAAAATCAATCTGTGGA | qRT | 11 | Solyc01g092950 |
| MADS2-F | | | | |
| RT- | GGCTGCATGATCCTAAACCTTC | qRT | 12 | |
| MADS2-R | | | | |
| RT- | GCCAAGCTCTAATGATCCTGCT | qRT | 13 | Solyc01g097290 |
| IAA16-F | | | | |
| RT- | GCCGGTGGTGTTTTTCTGA | qRT | 14 | |
| IAA16-R | | | | |
| RT-Pistil- | GGACTTGTTTACTGCAAACCTTG | qRT | 15 | Solyc02g078100 |
| specific | | | | |
| extensin- | | | | |
| like | | | | |
| protein-F | | | | |
| RT-Pistil- | TGTCTTTTTGGTGTTGTTGCAC | qRT | 16 | |
| specific | | | | |
| extensin- | | | | |
| like | | | | |
| protein-R | | | | |
| sg-7- | TAGGTCTCGACAGATCAACTGGTTTTAGA | CRISPR | 17 | |
| S1RF8AB- | GCTAGAA | | | |
| F | | | | |
| sg-7- | CGGGTCTCACTGTCAACTCCATGCACCAGC | CRISPR | 18 | |
| S1ARF8AB- | CGGG | | | |
| R | | | | |
| sg-8- | TAGGTCTCGTAGGCACACCAGGTTTTAGA | CRISPR | 19 | |
| S1ARF8AB- | GCTAGAA | | | |
| F | | | | |
| sg-8- | CGGGTCTCACCTACCAACTGTTGCACCAGC | CRISPR | 20 | |
| S1ARF8AB- | CGGG | | | |
| R | | | | |

### Example 1

### Tomato Class a SIARFs are Differentially Expressed in Multiple Flower Organs

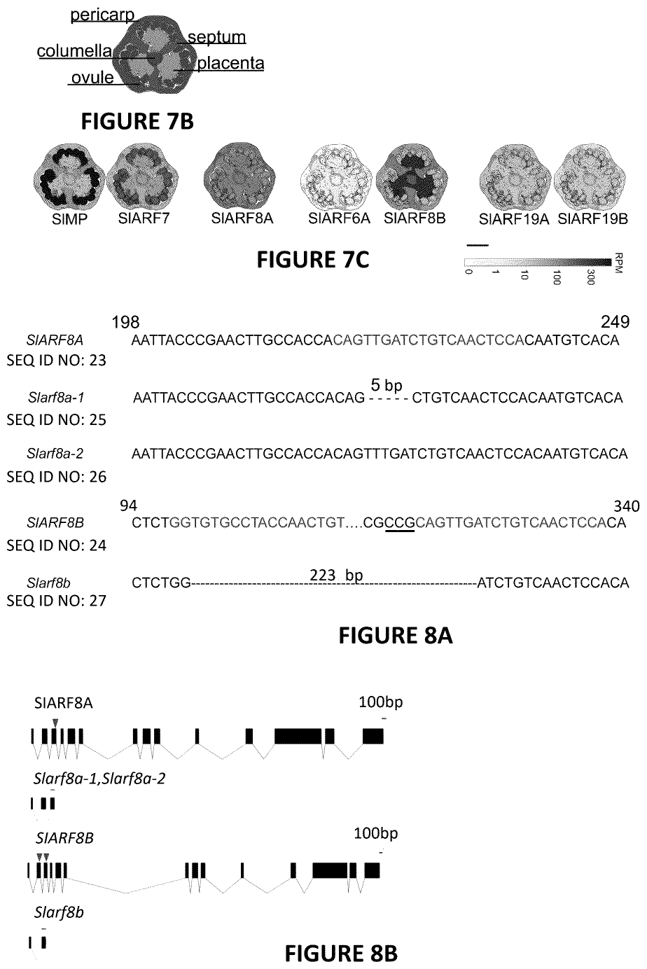

To explore the contribution of tomato class A ARFs to the control of fruit set and development, the inventors first examined their expression in ovary and fruit tissues. Analysis of RNAseq data revealed that all class A SlARFs are expressed in young ovaries five days before anthesis, with SlAMFA and SlAMFB expressed at the highest relative levels (FIG. 7A). In a public transcriptomic dataset obtained from micro-dissected ovary tissues at the day of anthesis, SlARF5/MP, M1AMFA and SlAMFB were the most highly expressed class A ARFs (Shinozaki et al., 2018). Of these, SlAMFA was expressed in the placenta and pericarp, and SlAMFB was particularly highly expressed in the placenta (FIG. 7B-7C). SlARF5/SlMP and SlARF7 were expressed mainly in ovules, and SlARF19A and SlARF19B expression was relatively low and uniform throughout the ovary. SlARF6A was expressed most strongly in the placenta but at much lower level than SlAMFA and SlAMFB. These expression patterns suggest that different class A SlARFs may regulate distinct aspects of fruit set and seed development, and that S1ARF8A and S1ARF8B may be particularly important for growth and development of the placenta, septum, and pericarp, which grow substantially when fruits form.

### Example 2

### Slarf8a Slarf8b Double Mutants Produce Parthenocarpic Fruits

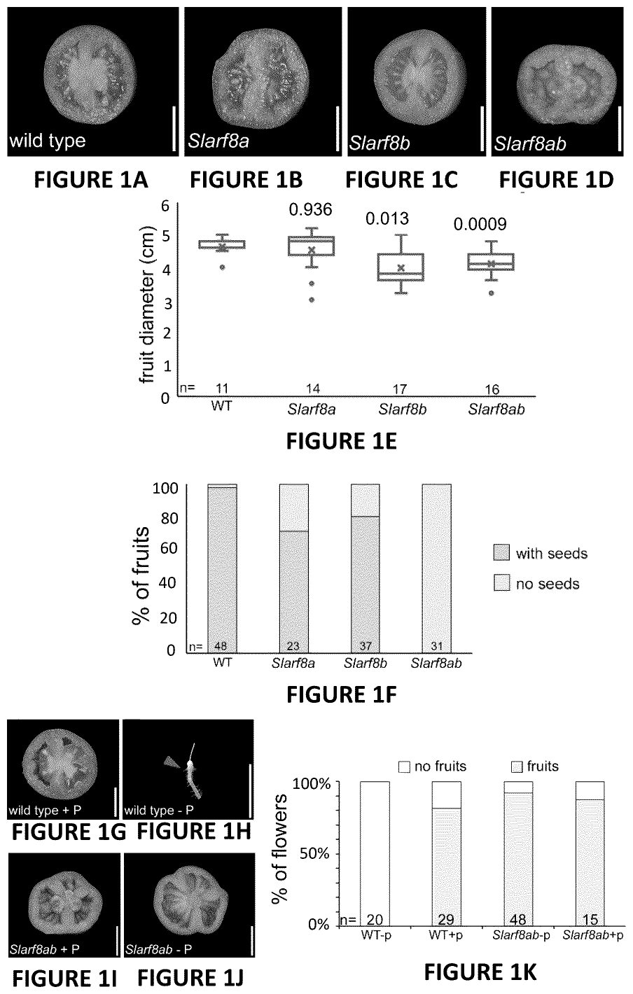

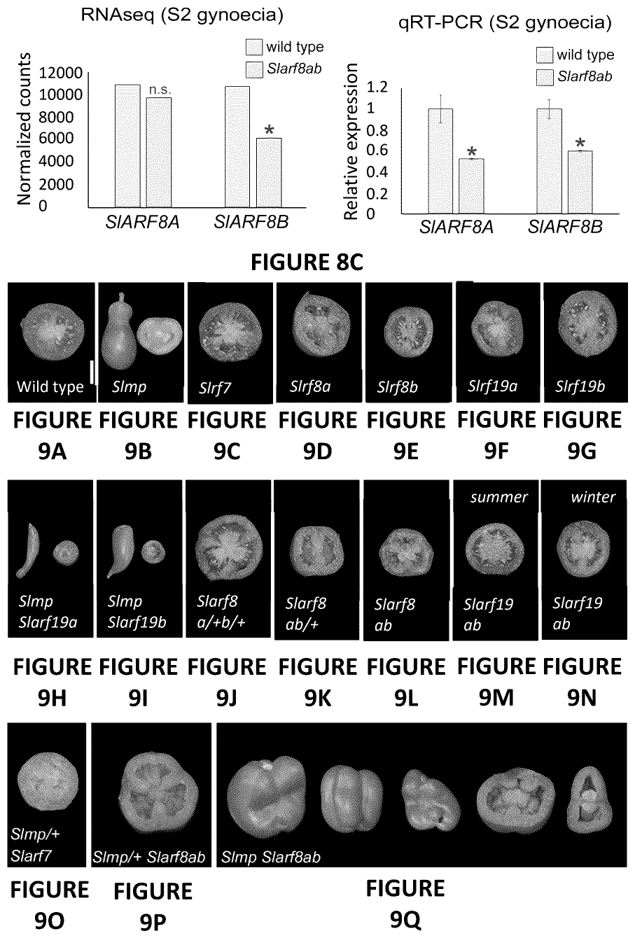

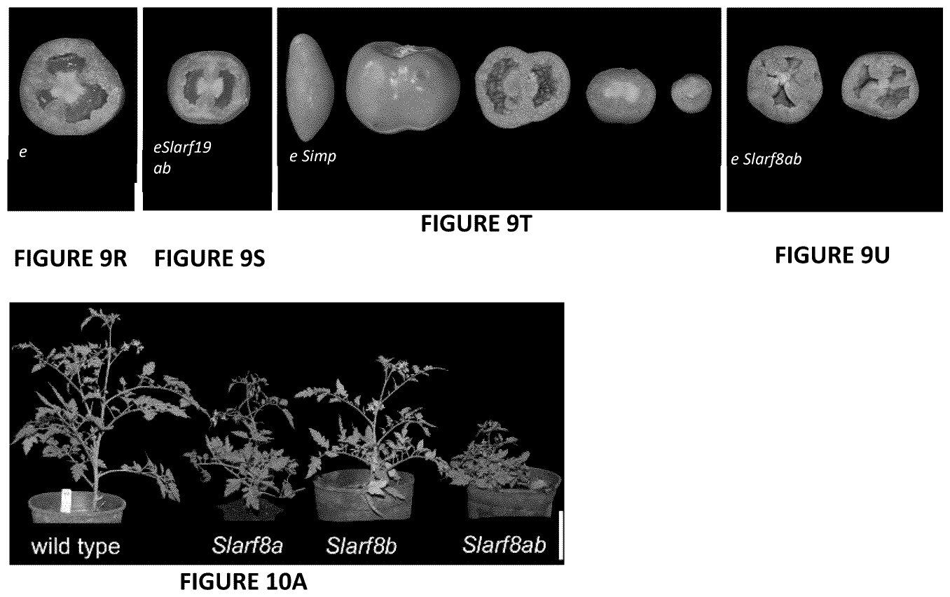

To elucidate the function of specific tomato class A ARFs in the control of fruit set, the inventors examined fruit development in single mutants or mutant combinations in this family. The inventors generated several mutant alleles in S1ARF8A and S1ARF8B (FIG. 8, see materials and methods), and used the previously described mutants in SlARF5/MP, SlARF7, SlARF19A and SlARF19B (Israeli et al., 2019). Slarf5/Slmp mutants and mutant combinations that contained it rarely developed fruits when allowed to self-pollinate, and these fruits were small, seedless, elongated and ovate-like (van Knaap et al., 2014) (FIGS. 9B, 9H-9I, 90, 9Q, and 9T and (Israeli et al., 2019)). Other single Slarf mutants did not have an apparent defect in fruit development. Of the double mutants, two combinations, Slarf8a Slarf8b and Slarf19a Slarf19b, consistently produced seedless fruits when allowed to self-pollinate (FIGS. 1, and 9N). Slarf8b and Slarf8a Slarf8b, but not Slarf8a, fruits were slightly smaller than wild-type fruits (FIGS. 1, and 9). While single Slarf8a and Slarf8b mutants showed low percentages of seedless fruit formation, all the fruits produced by Slarf8a Slarf8b double mutant plants were seedless, and mostly lacked locular gel and placenta (FIGS. 1A-1F, 9D-9E, and 9L).

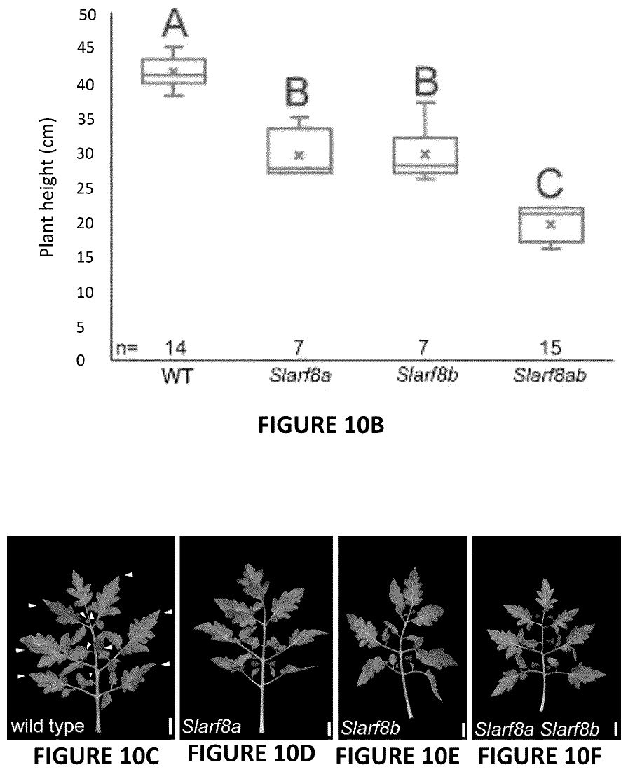

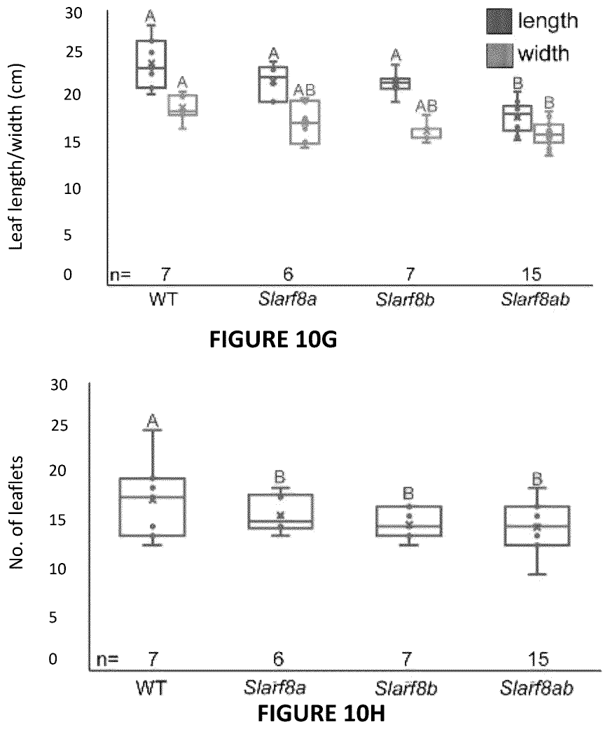

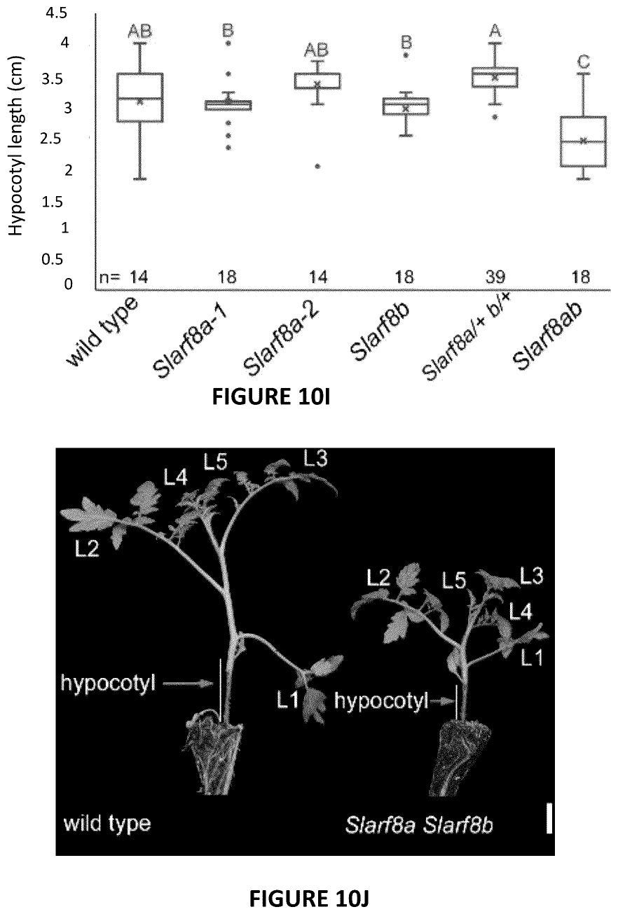

Previously described Slarf mutations affect different aspects of vegetative phenotypes during plant development (Israeli et al., 2019). The inventors inquired whether Slarf8a and Slarf8b mutant combinations also affect vegetative development. Slarf8 mutant plants were smaller with smaller and slightly fewer compound leaves compared to wild-type plants (FIGS. 10A-10H). The hypocotyls of Slarf8a Slarf8b were slightly shorter compared to the wild-type (FIGS. 9I-9J). Thus, S1ARF8A and B promote vegetative growth, in contrast to their effect on unpollinated ovary growth.

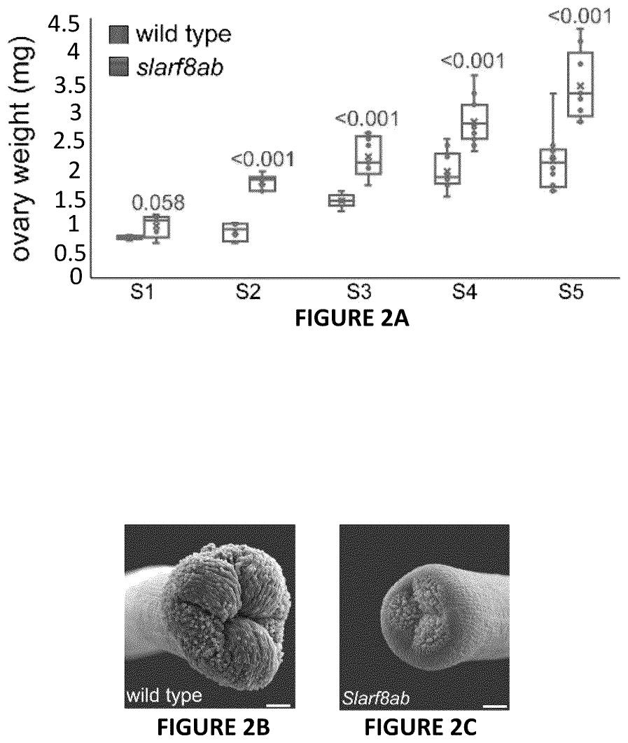

The specific placenta expression and the mutant phenotypes suggested that S1ARF8A and S1ARF8B are particularly relevant for fruit set in tomato. The inventors therefore focused further analysis mainly on Slarf8a and Slarf8b mutant combinations. The formation of seedless fruits is not always linked to the ability to form fruits independently of fertilization, termed parthenocarpy. To test for parthenocarpic fruit formation, the inventors therefore emasculated flowers before anthesis. In contrast to the wild-type, Slarf8a Slarf8b flowers produced parthenocarpic fruits following emasculation (FIGS. 1G-1K). To understand the developmental basis for this parthenocarpy, the inventors followed ovary development from early stages of flower development. The flower bud stages were classified according to bud and gynoecia size, opening of the sepals, color of the petals and opening of the flowers, according to (Dobritzsch et al., 2015). The youngest stage, designated stage 1 (S1) represented a small bud completely enclosed by sepals, seven days before anthesis. S2-S4 were buds 5, 3, and 1 days before anthesis, respectively, where S2 corresponds to stage 9-11 in (Brukhin et al., 2003). S5 was at the time of anthesis (flower opening), and S6 represented an open flower with bright yellow petals, one day after anthesis and pollination. Developing Slarf8a Slarf8b ovaries started growing earlier and grew faster than wild-type ovaries, starting from S2, and unlike the wild-type, did not pause growth at the S4 stage (FIG. 2A). Therefore, S1ARF8A and S1ARF8B appear to repress fruit set in unpollinated flowers, from a very early stage of flower development.

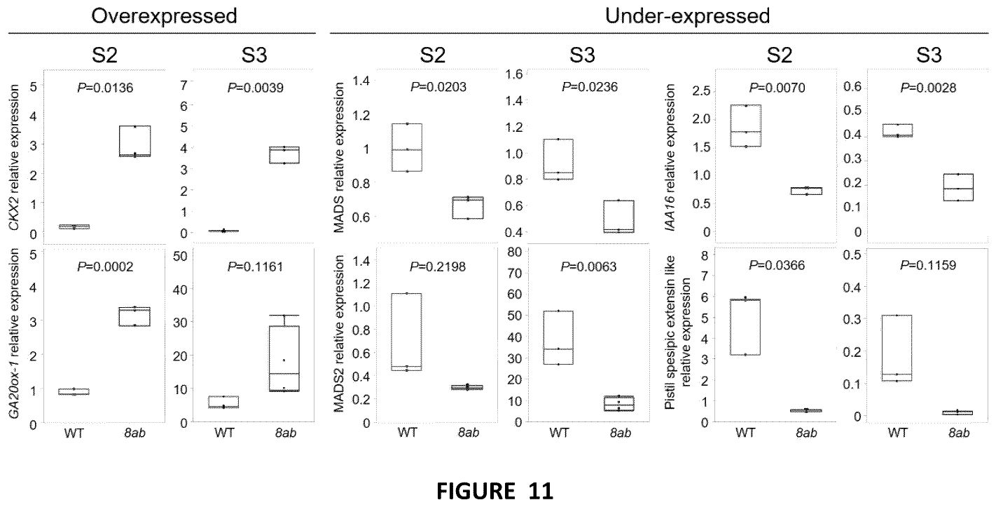

To better understand the role of S1ARF8A and B in ovary growth and inhibition of fruit set, the inventors compared gene expression between developing ovaries of wild-type and Slarf8a Slarf8b plants. Young S2 gynoecia, before major changes in weight and size could be observed, were used for RNAseq. This experiment was repeated twice. Among the genes commonly overexpressed in both experiments was the GA biosynthesis gene SlGA20ox-1 (Solyc03g006880), in agreement with GA acting downstream of auxin in fruit formation (Dorcey et al., 2009; Serrani et al., 2010), and with the finding that over-expression of SlGA20ox-1 leads to parthenocarpic fruit formation (Garcia Hurtado et al., 2012). The cytokinin degradation gene CKX2 (Solyc 10g017990) was also overexpressed Slarf8a Slarf8b (FIG. 11). Cytokinin was shown to promote fruit development (Bartrina et al., 2011, Joldersma and Liu, 2018), and the upregulation of CKX2 could result from feedback regulation or from a dual role for cytokinin in different stages of fruit development. The under-expressed genes included two MADS-BOX genes (SolycO1g092950/SlMADS2 and Solyc087990), implicated in the control of fruit set (Joldersma and Liu, 2018), the auxin-responsive gene SlIAA16 (SolycO1g097290), and a pistil-specific extensin-like protein (Solyc02g078100). Interestingly, several of the genes affected by Slarf8a Slarf8b, including SlGA20ox-1, Solyc087990 and Solyc02g078100, were similarly affected by natural or parthenocarpic fruit set (Tang et al., 2015). The inventors validated the effect of Slarf8a Slarf8b on the expression of several of the identified genes in two stages of gynoecium development, S2 and S3. In most cases, the effect was also apparent in the S3 stage (FIG. 11). The inventors also compared the DEG from the data with the DEG obtained from a related published dataset, from S. pimpinellifolium plants overexpressing miR167a, which targets SpARF6 and SpARF8 genes (Liu et al., 2014). Thirty-eight (38) genes were under-expressed in both Slarf8a Slarf8b and 35S:MIR167a, including SlIAA16. Forty genes (40) were overexpressed in Slarf8a Slarf8b and 35S:miR167a. These results suggest that class A SlARFs affect several pathways that are central to fruit formation and development, and may help in the future to identify putative class A S1ARF targets that mediate the effect of auxin on fruit set.

### Example 3

### SIARF8A and SIARF8B are Required for Jasmonate Production and Female Fertility

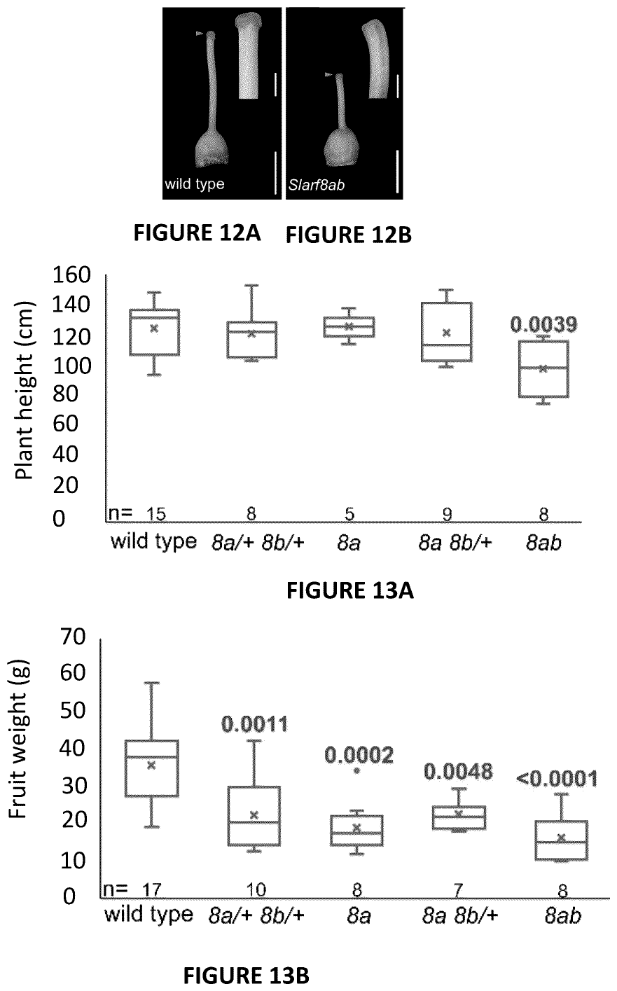

To understand why Slarf8a Slarf8b plants do not produce seeds, the inventors explored flower function and anatomy. In reciprocal pollinations, Slarf8a Slarf8b double mutants produced viable pollen but were female-sterile, similarly to S. pimpinellifolium p35S:AtMIR167a plants described previously (Liu et al., 2014). Stereo microscopy and scanning electron microscopy (SEM) revealed that Slarf8a Slarf8b mutant styles were shorter than wild-type styles, and that mutant stigmas had very few papillae and were shorter and narrower than wild-type stigmas (FIGS. 2B-2C, and 12).

### Example 4

### Slarf8a and Slarf8b Mutations Increase Yield Stability Under Non-Optimal Conditions

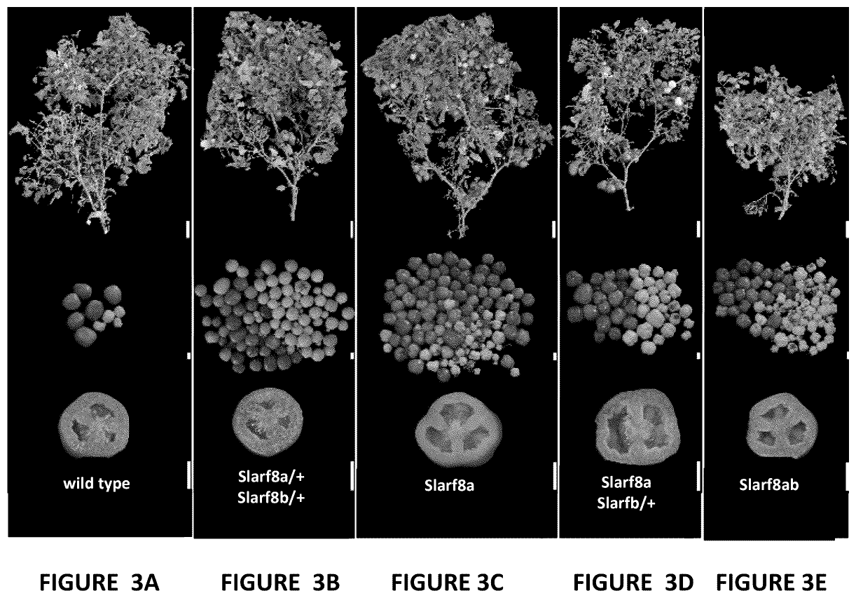

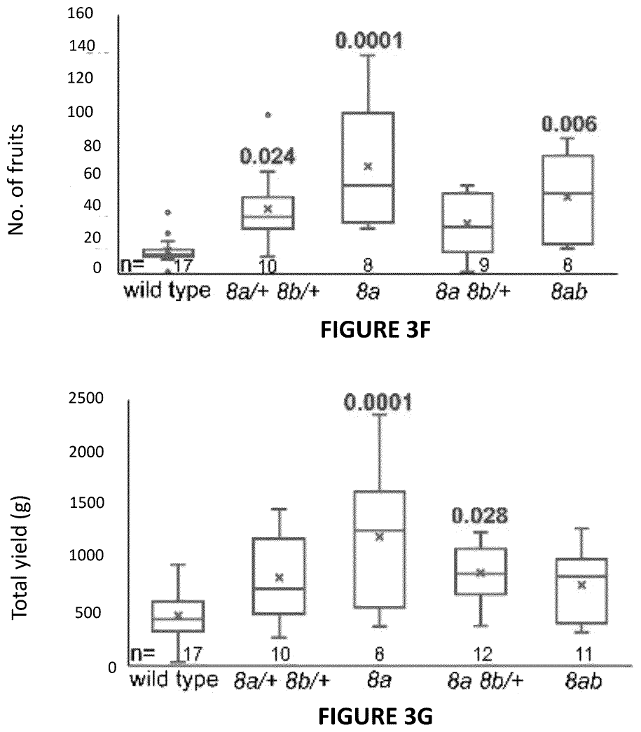

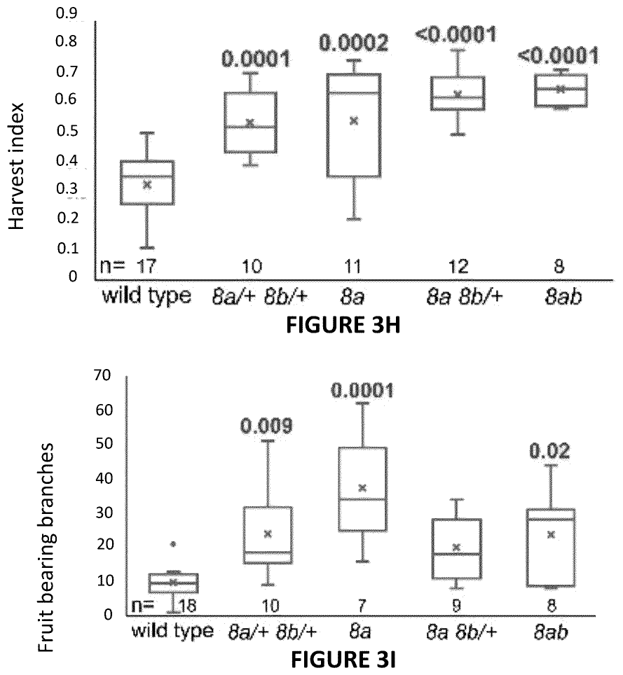

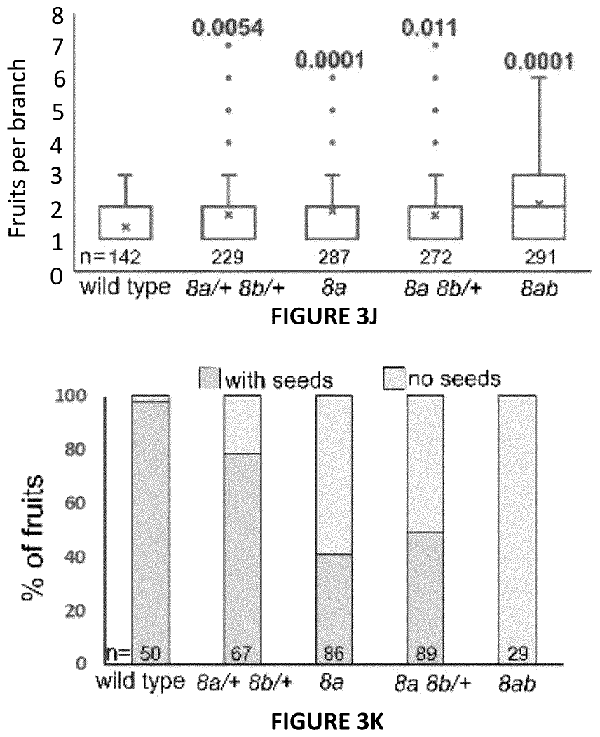

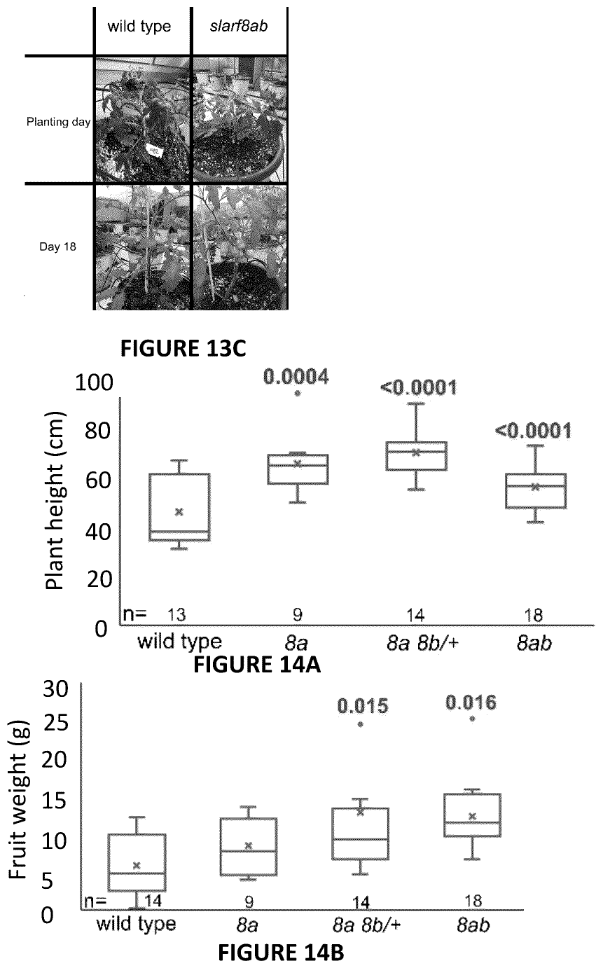

Extreme temperatures lead to yield loss and reduced fruit set (Charles & Harris, 1976; Wahid et al., 2007; Alsamir et al., 2021). The parthenocarpic and seedless phenotype of Slarf8a Slarfb double mutants and the partial seedless phenotypes of single Slarf8a and Slarf8b mutants (FIGS. 1-2, and 9) prompted us to test whether altering S1ARF8 activity can increase yield in extreme temperatures. The inventors hypothesized that an intermediate dosage of these paralogous genes might enable at once high-quality fruit and higher yield stability. The inventors therefore grew wild-type, Slarf8a/+ Slarf8b/+, Slarf8a, Slarf8a Slarf8b/+ and Slarf8a Slarf8b in a greenhouse with controlled hot temperatures (32° C. Day/28° C. night) and tested their growth and yield performance. The plants were allowed to self-pollinate. Under these conditions, wild-type plants produced a very low number of fruits (FIGS. 3A, and 3F-3G). In contrast, most of the Slarf8 mutant combinations produced higher fruit number and had higher total yield than the wild-type (FIGS. 3A-3G). Harvest index, the ratio of total fruit yield to total plant weight, is an important agronomical trait that indicates the efficiency of fruit production (Kwon et al., 2020). All the Slarf mutant combinations had significantly and substantially higher harvest index than the wild-type. Slarf8a Slarf8b had the highest harvest index, due to the combination of its compact plant habit and high yield (FIG. 3H). While Slarf8a Slarf8b fruits did not grow placenta with locular gel, Slarf8a and Slarf8a/+ Slarf8b/+ had more locular gel. Plants with reduced SlARF8 had slightly smaller fruits (FIG. 13) and produced more seedless fruits (FIG. 3K). Therefore, Slarf mutations can increase yield stability under hot conditions by relaxing the control of fruit set. Importantly, under controlled normal growth conditions, or under ambient conditions, yield of the different Slarf8 mutant combinations was similar to that of the wild-type, except some combinations that had slightly increased yield.

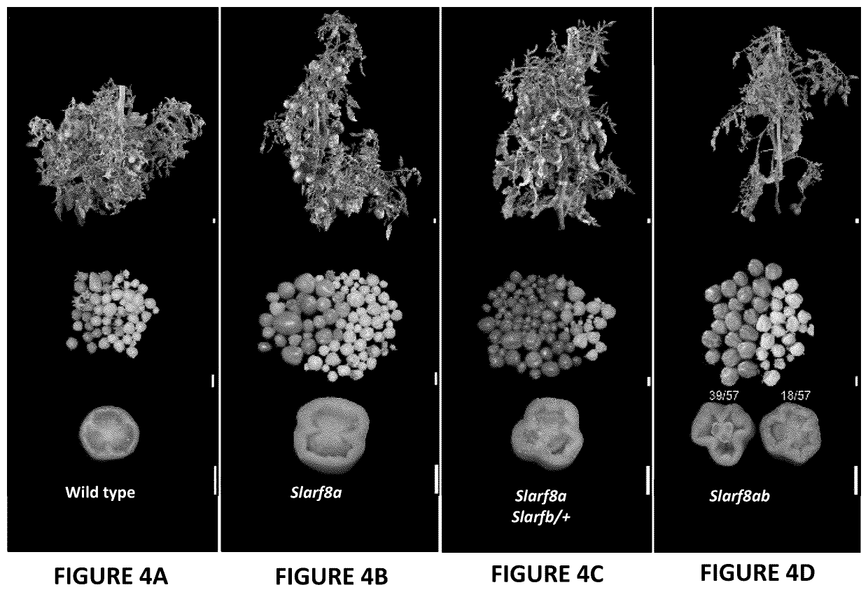

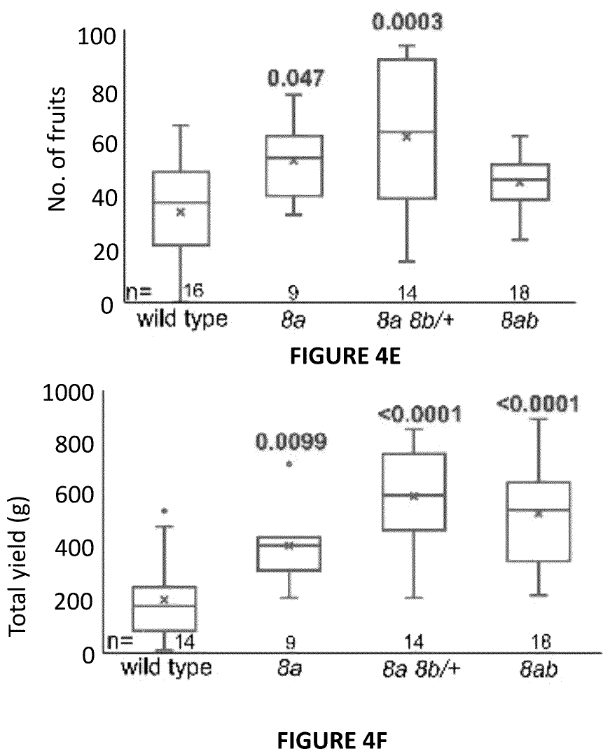

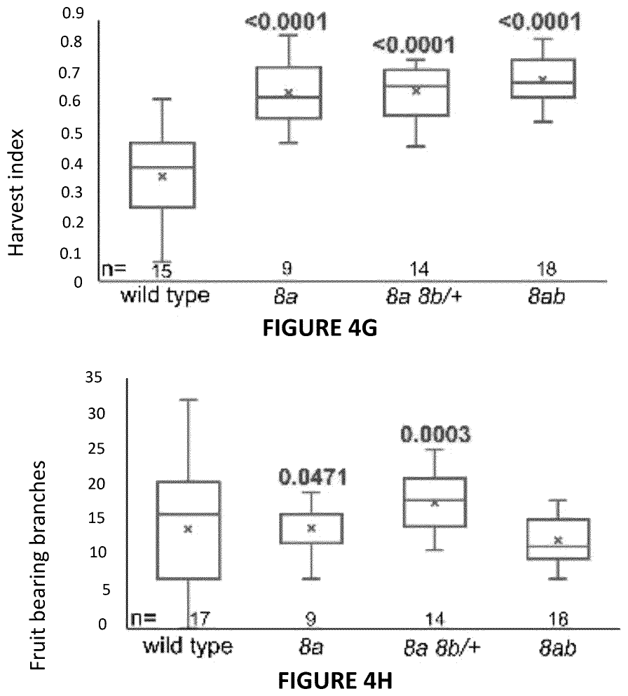

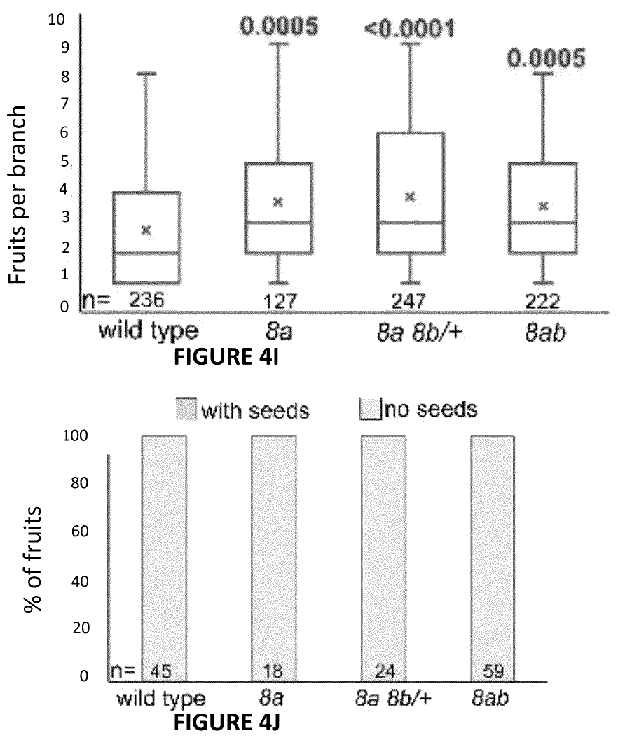

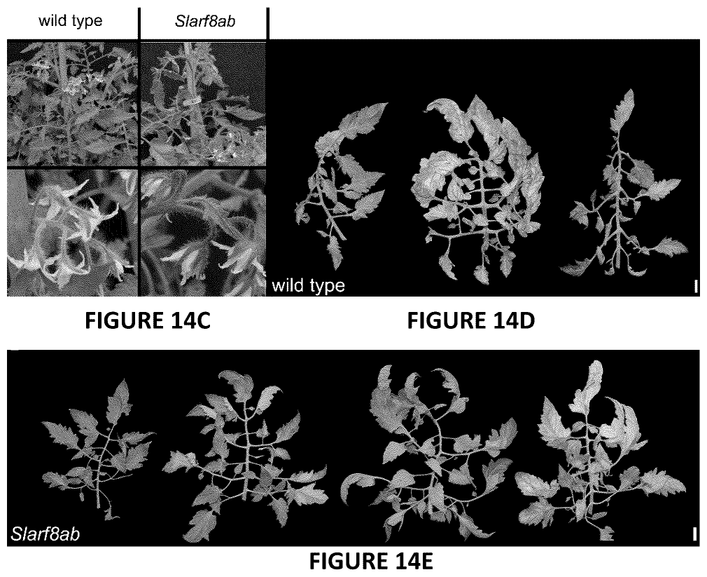

Extreme cold temperatures have also been reported to affect yield (Charles & Harris, 1976). The inventors therefore tested whether the Slarf8a and Slarf8b mutant combinations can increase yield also under cold-temperature stress (16° C. day/10° C. night). The plants were allowed to self-pollinate. Most of the Slarf8a and Slarf8b mutant combinations produced more fruits and had a higher yield and higher harvest index than the wild-type (FIGS. 4A-4G). This indicates that reduced S1ARF activity relaxes the control on fruit set also in the cold (FIGS. 4I, and 14). In addition to the effect on yield, leaves of Slarf8a Slarf8b double mutants remained green and appeared healthy in the cold when wild-type leaves had turned yellow and/or purple, suggesting higher general resistance to the cold (FIG. 14). Interestingly, all genotypes including the wild-type produced only seedless fruits in the cold (FIGS. 4A-4D, and 4J).

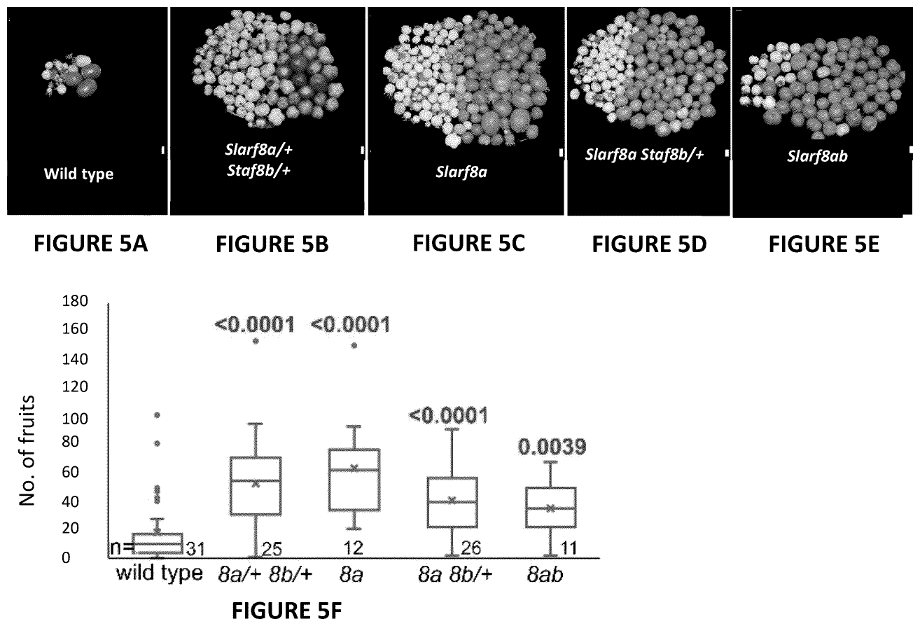

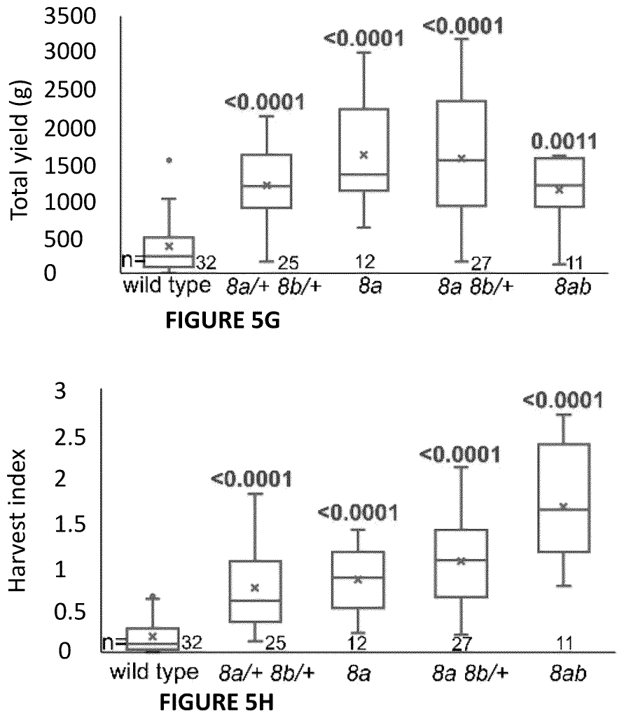

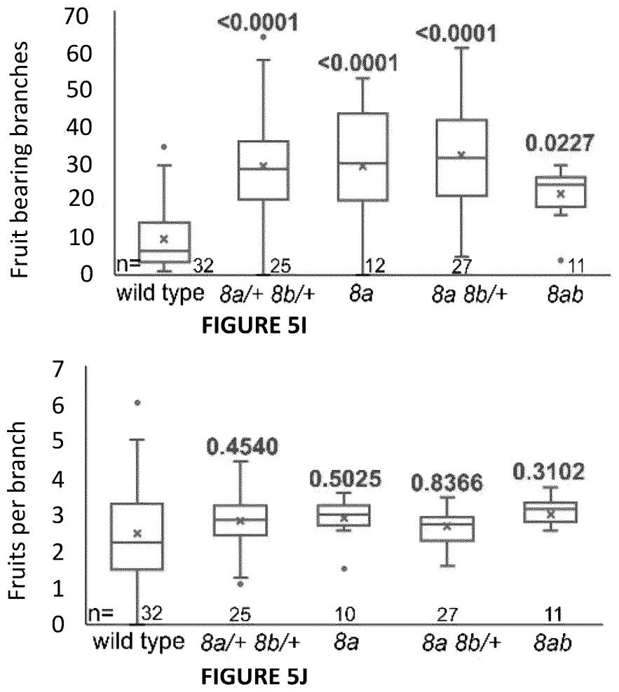

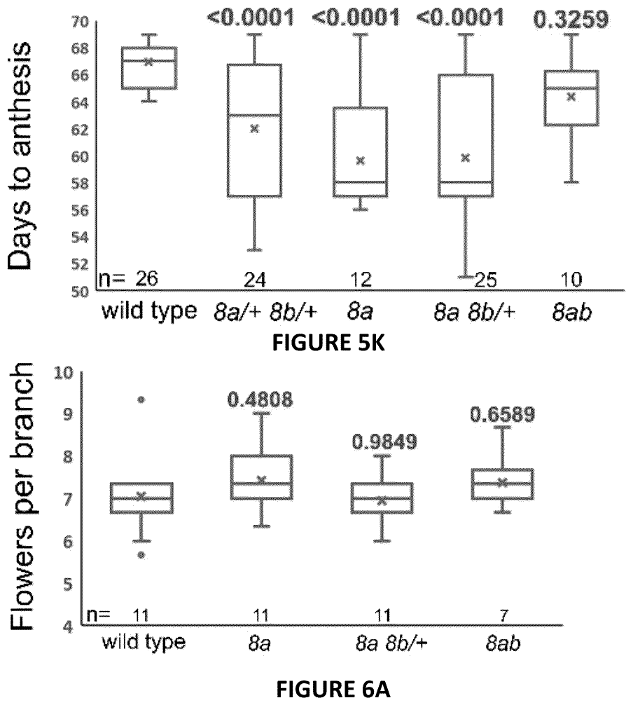

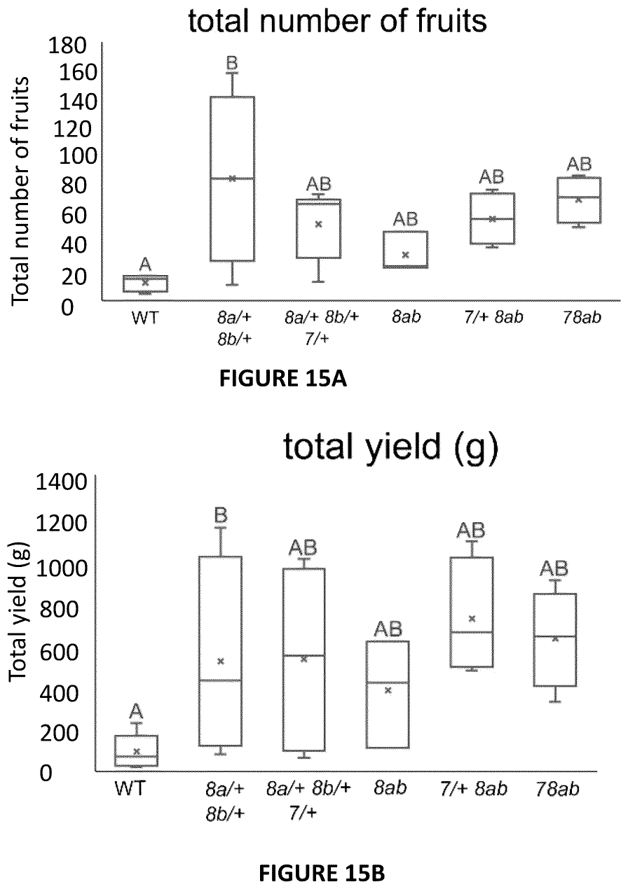

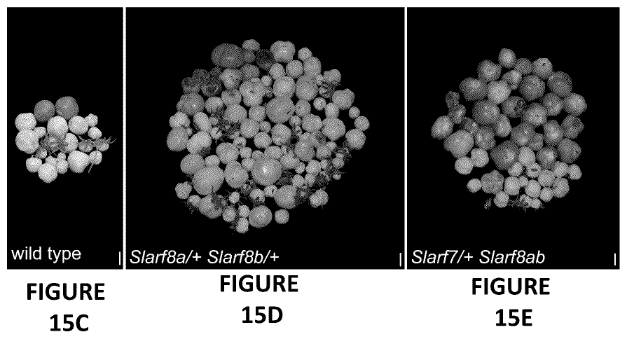

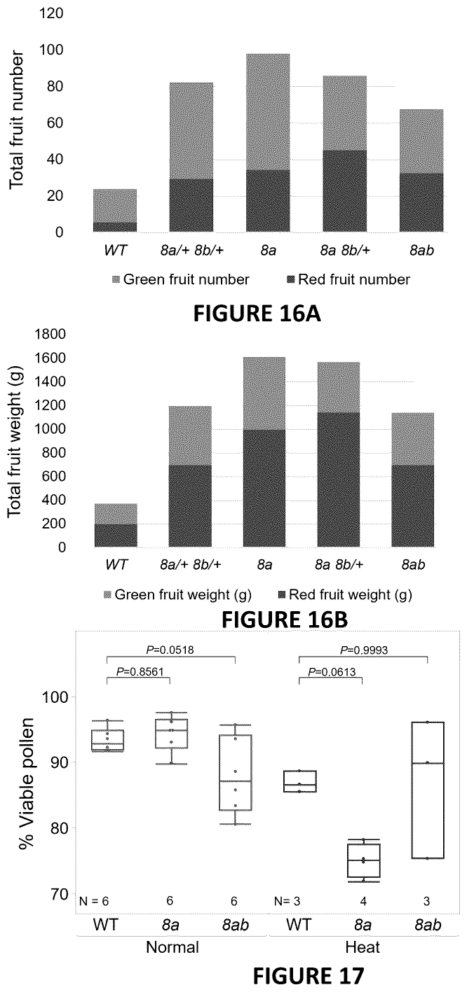

The experiments in controlled heat and cold stress conditions suggested that mutations in S1ARF8A and S1ARF8B might increase yield stability under extreme temperature stress. The inventors therefore tested the yield performance of the different Slarf8 mutant combinations under ambient heat stress conditions. An initial field test was performed with several mutant combinations. In this experiment, plants were grown outdoors in the field in Rehovot, Israel, between March and August 2019, during which they experienced several heat episodes. Under these conditions, wild-type plants produced few small fruits, while plants carrying mutant alleles of Slarf8a and Slarf8b had increased fruit number and weight (FIG. 15). In agreement with the experiments in controlled conditions, the inventors observed an effect for some heterozygous mutant alleles. The inventors therefore performed a second experiment with several genotypes with a gradually reduced SlARF8 dose. Plants were grown in a net-house in the soil under field conditions, with no temperature control, in Rehovot, Israel, between May and August 2021, during which they experienced temperatures of above 40° C. for 3-5 hours every day, for a period of several weeks. The plants were allowed to self-pollinate. The wild-type plants had a very low number of fruits. In contrast, all of the Slarf8 mutant combinations had a substantially and significantly higher number of fruits, higher total fruit weight and higher harvest index (FIGS. 5A-5H, and 16). The best-performing genotype was Slarf8a, with more than 3-fold more fruits and more than 4-fold fruit weight relative to the wild-type. This suggests that partial reduction of Slarf8 dose bypasses the effect of temperature on yield. As vegetative growth and fruit appearance are normal in single Slarf8a mutants, they can be attractive for breeding purposes.

### Example 5

### Changing SlARF8 Dose Increases Yield Via Several Traits

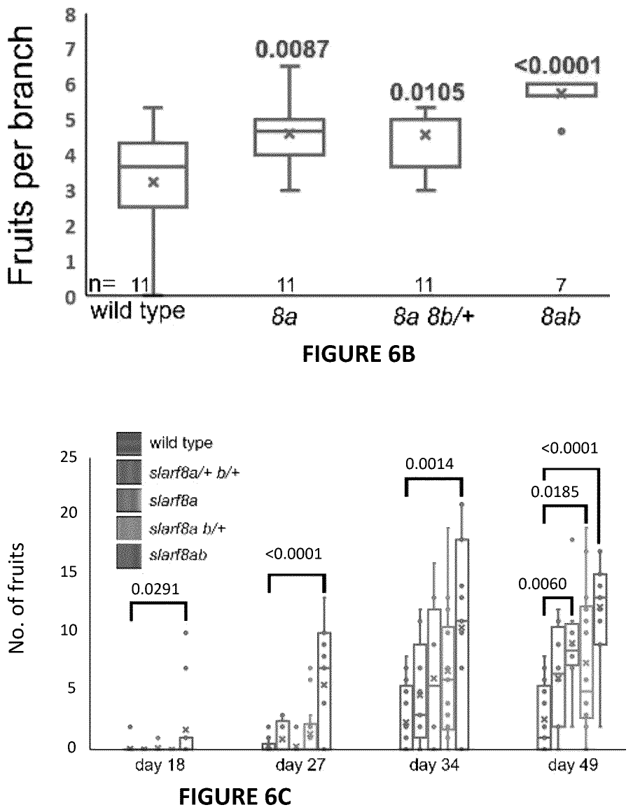

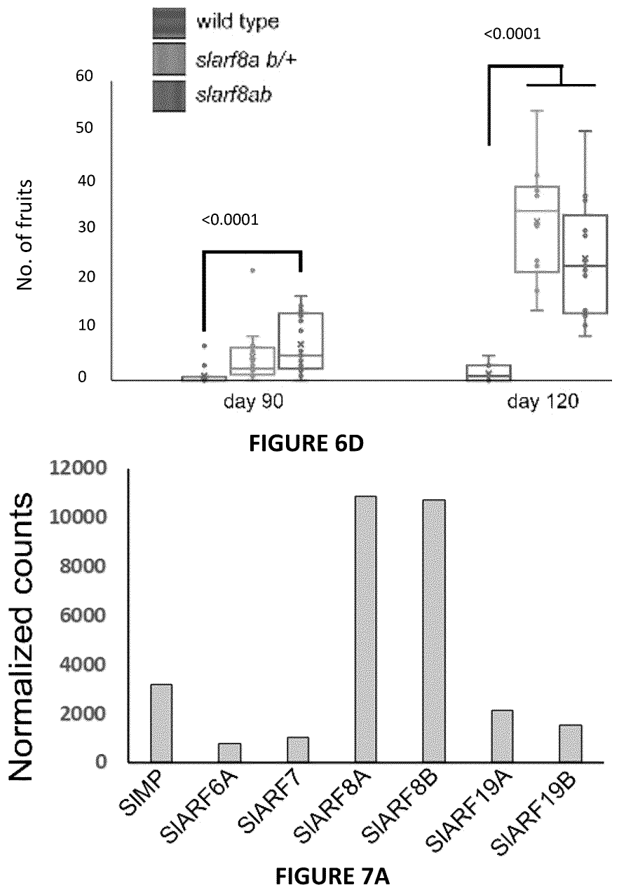

While Slarf8a Slarf8b double mutants were parthenocarpic and seedless, other mutant combinations with intermediate S1ARF8 dose were not parthenocarpic and were only partly seedless. Therefore, the effect of SlARF8s on fruit set appears to be more complex than just affecting parthenocarpy. The inventors therefore examined the developmental basis of the improved yield of Slarf8 mutants. Pollen viability assays under normal and controlled heat conditions showed no statistically significant difference between wild-type, Slarf8a and Slarf8a Slarf8b plants (FIG. 17). The inventors monitored flowering time, time of initial fruit production, number of fruit-bearing branches, and number of fruit per fruit-bearing branch in the different experiments under controlled or ambient temperature stress. In the second field experiment, Slarf8a, Slarf8a/+ Slarf8b/+ and Slarf8a Slarf8b/+ all flowered significantly and substantially earlier than the wild-type. Interestingly, Slarf8a Slarf8b double mutants flowered at the same time as the wild-type, suggesting a complex interaction between S1ARF8A and B with respect to flowering time (FIG. 5K). Most of the tested Slarf8 mutant combinations made substantially and statistically significantly more fruit-bearing branches than the wild-type, in all tested temperature-stress conditions, although the extent differed in the different conditions (FIGS. 3I, 4H, and 5I). Average number of fruits per fruit-bearing branch was significantly higher in all mutant combinations in the controlled heat and cold conditions, but not in the field experiment (FIGS. 3J, 4I, and 5J). To further understand the basis for more fruits per branch, the inventors counted the number of flowers and the number of fruits on specific inflorescences. While all the tested genotypes produced a similar number of flowers per inflorescence, all tested Slarf8 mutant combinations produced more fruits per branch in comparison with the wild-type (FIGS. 6A-6B). This indicates that more flowers set fruit in the Slarf8 mutants. In the controlled heat and cold conditions, the different Slarf8 mutants started to produce fruits earlier than the wild-type (FIGS. 6C-6D, 13C, and 14C). Therefore, the substantially increased fruit number and fruit weight of the Slarf8 mutants results from a combination of earlier onset of fruit set, production of more fruit-bearing branches, and more flowers that produce fruit. The exact contribution of each component differs depending on the Slarf8 dosage and the external conditions. The current findings suggest that a possible approach to increase yield stability under unstable climate may be to breed plants that bypass the effects of variable weather to produce fruits under all conditions, analogous to breeding day-neutral flowering time for different latitudes. In particular, partially reducing the activity of a subset of class A ARFs, as provided herein, increases stable fruit set in an array of different environments. Thus, each genetic combination of Slarf8a and Slarf8b alleles (0-3 functional alleles) has a slightly different effect on flexibility and stability of fruit set. Moreover, in addition to their effects on fruit set, SlARF8a and SlARF8b genes also have dose-dependent effects on plant stature and on leaf size and shape. Slarf8a Slarf8b mutant plants produced fruit after emasculation and are therefore truly parthenocarpic, genotypes with a partial reduction in SlARF8 gene dosage, such as Slarf8a, Slarf8a/+ Slarf8b/+ and Slarf8a Slarf8b/+, produced some seedless fruits, though did not produce fruits after emasculation. Nevertheless, fruit set in these genotypes was equally robust in extreme temperatures compared to the fully parthenocarpic genotypes. Indeed, the inventors observed early flowering, more fruit-bearing branches and more flowers setting fruits in some of the S1ARF8-deficient genotypes, and these changes may contribute to overall fruit production. Each of these traits may be less sensitive to extreme temperatures in the mutants.

### Example 6

### Slarf8a and Slarf8b Mutations Increase Plant Resistance to Pathogens

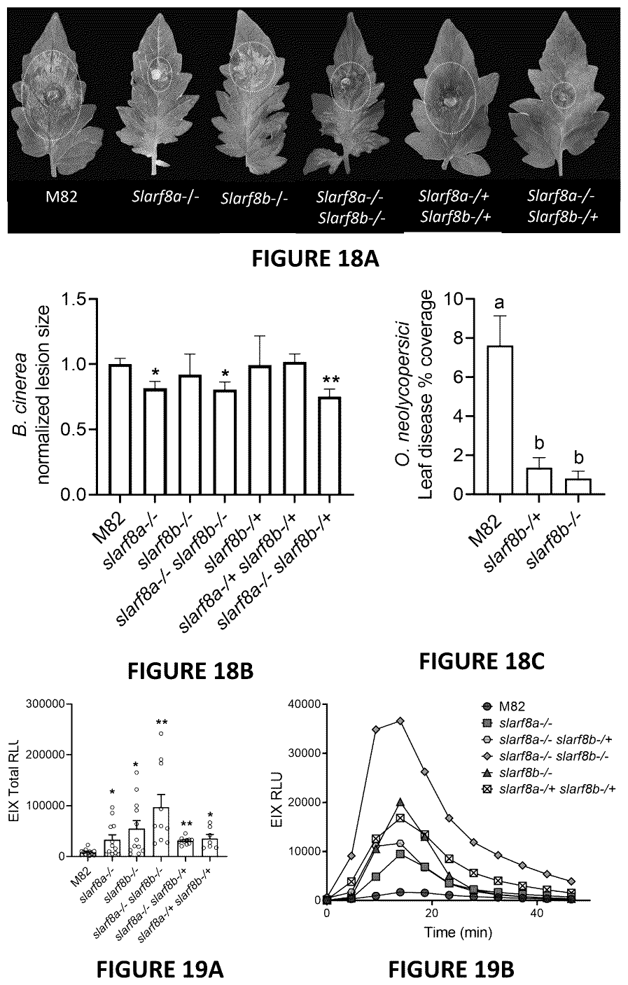

The inventors have further examined whether the SlARF8 genotypes disclosed herein are disease resistant. M82 tomato plants of different SlARF8 mutant genotypes were infected with 4-day old Botrytis cinerea mycelia. Disease was monitored after 4 days. The results show that all plants harboring two inactive ARF8a alleles were characterized by significantly increased resistance to B. cinerea (FIGS. 18A-18B). Further, the inventors have examined resistance of SlARF8b mutants to Oidium neolycopersici spores. Specifically, mutants were infected with 10⁴ O. neolycopersici spores, and disease was monitored after 10 days. The results show that mutant plants harboring either one or two inactive ARF8b alleles, are significantly more resistance to O. neolycopersici infection (18C).

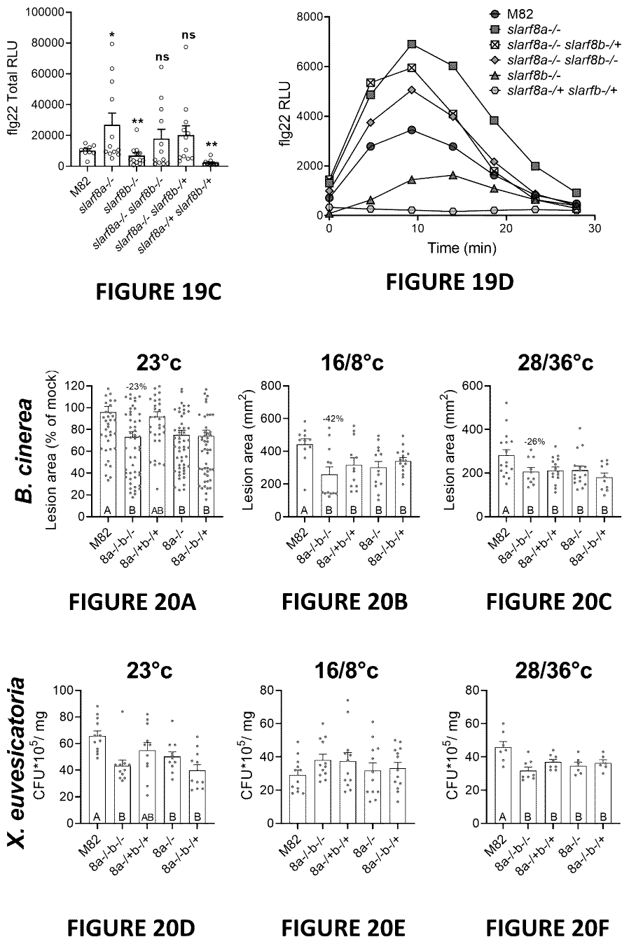

Further, the inventors have examined whether SlARF8 genotypes as disclosed herein have stronger immune responses. For this purpose, M82 tomato plants of different S1ARF8 mutant genotypes were challenged with any one of: the fungal elicitor ethylene-inducing xylanase (EIX), and the bacterial elicitor flg22. Total reactive oxygen species (ROS) produced, were determined and expressed in relative luminescent units (RLU). The results show that plants bearing at least one inactive allele of any one of ARF8a and ARF8b are characterized by a significantly stronger immune response to EIX, compared to control (FIGS. 19A-19B). To this end, plants harboring two inactive alleles of ARF8a were found to be characterized by a significantly stronger immune response to flg22, compared to control (FIGS. 19C-19D).

Further, the inventors have examined whether the SlARF8 genotypes disclosed herein, maintain disease resistance under extreme temperature conditions. For this purpose, M82 tomato plants of different SlARF8 mutant genotypes, grown under temperatures regimen as describe above, were infected with 4-day old B. cinerea mycelia or injected with 10⁶ CFU of X. euvesicatoria. Disease was monitored after 4 days, by measuring lesion area for B. cinerea, or plating serial dilutions of macerated tissue and quantifying bacterial load for X. euvesicatoria. The results show that plants harboring two inactive ARF8a alleles (and possibly further comprising one or two inactive ARFb8 allele(s) are characterized by significantly increased resistance to B. cinerea (compared to control), either in ambient temperature, or under extreme temperatures (FIGS. 20A-20C, respectively). Further, the results show that plants harboring two inactive ARF8a alleles (and possibly further comprising one or two inactive ARFb8 allele(s) are characterized by significantly increased resistance to X. euvesicatoria (compared to control), either in ambient temperature, or under extreme temperatures (FIGS. 20D-20F, respectively). Plants harboring at least one inactive ARF8a allele, and any one of: a second inactive ARF8a allele, at least one inactive ARF8b allele, two inactive ARF8b alleles, or any combination thereof, were also shown to retain their significantly increased resistance to X. euvesicatoria (compared to control) under extreme hot temperatures (FIG. 20F).

While the invention has been described in conjunction with specific embodiments thereof, it is evident that many alternatives, modifications, and variations will be apparent to those skilled in the art. accordingly, it is intended to embrace all such alternatives, modifications and variations that fall within the spirit and broad scope of the appended claims.

1.-19. (canceled)

20. A genetically modified Solanum plant comprising any one of: at least one inactive allele of ARF8a gene, at least one inactive allele ARF8b gene, and both, wherein said genetically modified Solanum plant comprises a genome being devoid of any one of: (i) one or both alleles of ARF8a gene comprising the nucleic acid sequence: ATGAAGCTTTCAACATGGAATGGGTCCAGCAAGCTCATGA (SEQ ID NO: 29), ATGAAGCTTTCCATCAGGAATGGGTCCAGCAAGCTCATGA (SEQ ID NO: 30), or both; (ii) one or both alleles of ARF8b gene comprising the nucleic acid sequence: ATGAAGCTTTCAACATCAGAGAATGGGTCAGCAGGCTCATGA (SEQ ID NO: 31), ATGAAGCTTTCTCAGGAATGGGTCAGCAGGCTCATGAAGGAGGAGAGAAAA AGTGTTTGA (SEQ ID NO: 32), or both; or (iii) both (i) and (ii).

21. The genetically modified plant of claim 20, wherein said genetically modified Solanum plant comprising at least one inactive allele of the ARF8a gene comprising the nucleic acid sequence selected from:
(SEQ ID NO: 25)
AATTACCCCGAACTTGCCACCACAGCTGTCAACTCCACAATGTCAC;
or
(SEQ ID NO: 26)
AATTACCCCGAACTTGCCACCACAGTTTGATCTGTCAACTCCACAATGT
CAC.

22. The genetically modified Solanum plant of claim 20, comprising at least one inactive allele of the ARF8b gene comprising the nucleic acid sequence: CTCTGGATCTGTCAACTCCACA (SEQ ID NO: 27).

23. The genetically modified Solanum plant of claim 20, comprising: (i) at least one inactive allele of the ARF8a gene comprising the nucleic acid sequence set forth in SEQ ID NO: 25 and at least one inactive allele of the ARF8b gene comprising the nucleic acid sequence set forth in SEQ ID NO: 27; or (ii) at least one inactive allele of the ARF8a gene comprising the nucleic acid sequence set forth in SEQ ID NO: 26 and at least one inactive allele of the ARF8b gene comprising the nucleic acid sequence set forth in SEQ ID NO: 27.

24. The genetically modified Solanum plant of claim 20, wherein both alleles of any one of: said ARF8a gene, said ARF8b gene, and both, are inactive.

25. The genetically modified Solanum plant of claim 20, comprising: (i) two inactive alleles of the ARF8a gene comprising the nucleic acid sequence set forth in SEQ ID NO: 25 and two inactive alleles of the ARF8b gene comprising the nucleic acid sequence set forth in SEQ ID NO: 27; or (ii) two inactive alleles of the ARF8a gene comprising the nucleic acid sequence set forth in SEQ ID NO: 26 and two inactive alleles of the ARF8b gene comprising the nucleic acid sequence set forth in SEQ ID NO: 27.

26. The genetically modified Solanum plant of claim 20, being characterized by: (i) increased yield; (ii) increased resistance to a pathogen; (iii) earlier fruit setting; and (iv) any combination of (i) to (iii), compared to a wild-type variant of said Solanum plant.

27. The genetically modified Solanum plant of claim 26, wherein said increased yield is under temperature stress.

28. The genetically modified Solanum plant of claim 27, wherein said temperature stress is heat stress, cold stress, or both.

29. A method for selecting an improved genetically modified Solanum plant, the method comprising:
a. determining the presence of at least one inactive allele of auxin responsive factor (ARF) 8 gene in the genome of said genetically modified Solanum plant or a part derived therefrom; and
b. selecting a genetically modified Solanum plant determined as having a genome comprising said at least one inactive allele of ARF8 gene, wherein said improvement is at least any one of: (i) increased yield; (ii) increased resistance to a pathogen; (iii) earlier fruit setting; and (iv) any combination of (i) to (iii), compared to a wild-type variant of said Solanum plant,
thereby, selecting an improved genetically modified Solanum plant.

30. The method of claim 29, wherein said increased yield is under culture conditions being sub-optimal for culturing said wild-type variant of said Solanum plant, and optionally wherein said sub-optimal conditions comprise: heat sub-optimal conditions, cold sub-optimal conditions, or both.

31. The method of claim 29, further comprising a step preceding said step (a), comprising producing said genetically modified Solanum plant, wherein said producing comprises contacting a Solanum plant or a part derived therefrom with an effective amount of an agent capable of inactivate any one of said allele of ARF8 gene, a transcript thereof, a protein product thereof, and any combination thereof, in said Solanum plant or a part derived therefrom, thereby producing said genetically modified Solanum plant, and optionally wherein: (i) said agent comprises a polynucleotide, a protein, or both, being a clustered regularly interspaced short palindromic repeats (CRISPR) system; (ii) said agent comprises at least one single guide RNA (sgRNA) configured to targeting said ARF8 gene, and a CRISPR associated (Cas) protein; or both (i) and (ii).

32. The method of claim 29, wherein said at least allele of said ARF8 gene being inactive is: knocked out, mutated, or knocked down, wherein an mRNA transcribed therefrom, a protein product translated therefrom, or both, is inactive or absent, in said genetically modified plant.

33. The method of claim 29, wherein two alleles of said ARF8 gene being inactive are: knocked out, mutated, or knocked down, wherein an mRNA transcribed therefrom, a protein product translated therefrom, or both, is inactive or absent, in said genetically modified plant.

34. The method of claim 29, wherein said Solanum plant is characterized by having a genome comprising at least two paralogs of ARF8 gene, and optionally wherein said at least two paralogs of ARF8 gene comprise ARF8a gene and ARF8b gene.

35. The method of claim 32, wherein said genetically modified Solanum plant comprises a genome comprising at least one allele of any one of ARF8a gene, ARF8b gene, or both, being: knocked out, mutated, or knocked down, such that an mRNA transcribed therefrom, a protein product translated therefrom, or both, is inactive or absent, in said genetically modified plant.

36. The method of claim 32, wherein said genetically modified Solanum plant comprises a genome comprising two alleles of any one of ARF8a gene, ARF8b gene, or both, being: knocked out, mutated, or knocked down, such that an mRNA transcribed therefrom, a protein product translated therefrom, or both, is inactive or absent, in said genetically modified plant.

37. The method of claim 29, wherein said genetically modified Solanum plant is a Solanum lycopersicum (tomato) plant.

38. The method of claim 29, further comprising a step proceeding step (b), comprising culturing said selected genetically modified Solanum plant of step (b).

39. The method of claim 29, wherein said yield comprises at least one parameter being selected from the group consisting of: number of fruit, total yield (gr), harvest index, number of inflorescences, number of fruit per inflorescence, % of parthenocarpic fruit, and any combination thereof, of said genetically modified plant.


SEQUENCE LISTING (33 sequences)

SEQ ID NO: 1 - DNA, 21 bp, synthetic construct
  source 1..21: mol_type = other DNA
tgacatcgaatggaaattcag

SEQ ID NO: 2 - DNA, 22 bp, synthetic construct
  source 1..22: mol_type = other DNA
gtctcttagcactaacaaacac

SEQ ID NO: 3 - DNA, 20 bp, synthetic construct
  source 1..20: mol_type = other DNA
gtcagtccgtgatcatagag

SEQ ID NO: 4 - DNA, 21 bp, synthetic construct
  source 1..21: mol_type = other DNA
ggaatccaagctacaatttcc

SEQ ID NO: 5 - DNA, 21 bp, synthetic construct
  source 1..21: mol_type = other DNA
tgctgctgtttttgtggacat

SEQ ID NO: 6 - DNA, 20 bp, synthetic construct
  source 1..20: mol_type = other DNA
ttctggtatcgtggctgaca

SEQ ID NO: 7 - DNA, 22 bp, synthetic construct
  source 1..22: mol_type = other DNA
agattgtgttggtggacttcaa

SEQ ID NO: 8 - DNA, 19 bp, synthetic construct
  source 1..19: mol_type = other DNA
tagcgccataaatgtgtcg

SEQ ID NO: 9 - DNA, 20 bp, synthetic construct
  source 1..20: mol_type = other DNA
ccggggagctttctgttctt

SEQ ID NO: 10 - DNA, 23 bp, synthetic construct
  source 1..23: mol_type = other DNA
ggcagaattatctgtcgaggcta

SEQ ID NO: 11 - DNA, 25 bp, synthetic construct
  source 1..25: mol_type = other DNA
aatctgatgaaaatcaatctgtgga

SEQ ID NO: 12 - DNA, 22 bp, synthetic construct
  source 1..22: mol_type = other DNA
ggctgcatgatcctaaaccttc

SEQ ID NO: 13 - DNA, 22 bp, synthetic construct
  source 1..22: mol_type = other DNA
gccaagctctaatgatcctgct

SEQ ID NO: 14 - DNA, 19 bp, synthetic construct
  source 1..19: mol_type = other DNA
gccggtggtgtttttctga

SEQ ID NO: 15 - DNA, 23 bp, synthetic construct
  source 1..23: mol_type = other DNA
ggacttgtttactgcaaaccttg

SEQ ID NO: 16 - DNA, 22 bp, synthetic construct
  source 1..22: mol_type = other DNA
tgtctttttggtgttgttgcac

SEQ ID NO: 17 - DNA, 36 bp, synthetic construct
  source 1..36: mol_type = other DNA
taggtctcgacagatcaactggttttagagctagaa

SEQ ID NO: 18 - DNA, 34 bp, synthetic construct
  source 1..34: mol_type = other DNA
cgggtctcactgtcaactccatgcaccagccggg

SEQ ID NO: 19 - DNA, 36 bp, synthetic construct
  source 1..36: mol_type = other DNA
taggtctcgtaggcacaccaggttttagagctagaa

SEQ ID NO: 20 - DNA, 34 bp, synthetic construct
  source 1..34: mol_type = other DNA
cgggtctcacctaccaactgttgcaccagccggg

SEQ ID NO: 21 - RNA, 20 nt, synthetic construct
  source 1..20: mol_type = other RNA
cagttgatctgtcaactcca

SEQ ID NO: 22 - RNA, 20 nt, synthetic construct
  source 1..20: mol_type = other RNA
tggagttgacagatcaactg

SEQ ID NO: 23 - DNA, 51 bp, Solanum lycopersicum
  source 1..51: mol_type = genomic DNA
aattacccgaacttgccaccacagttgatctgtcaactccacaatgtcaca

SEQ ID NO: 24 - DNA, 243 bp, Solanum lycopersicum
  source 1..243: mol_type = genomic DNA
ctctggtgtgcctaccaactgtcggaagtcgagttgtttactttcctcagggtcatagtgaacaggtctttctccttttcattctttatcacacaaactgatacaatctattattttagttataaatcactgatgttcccttactggaatggagtgacaggtagctgcaacaactaataaagaagttgatgcacatatacccaattacccaaatttgtcgccgcagttgatctgtcaactcca

SEQ ID NO: 25 - DNA, 46 bp, synthetic construct
  source 1..46: mol_type = other DNA
aattacccgaacttgccaccacagctgtcaactccacaatgtcaca

SEQ ID NO: 26 - DNA, 52 bp, synthetic construct
  source 1..52: mol_type = other DNA
aattacccgaacttgccaccacagtttgatctgtcaactccacaatgtcaca

SEQ ID NO: 27 - DNA, 22 bp, synthetic construct
  source 1..22: mol_type = other DNA
ctctggatctgtcaactccaca

SEQ ID NO: 28 - skipped

SEQ ID NO: 29 - DNA, 40 bp, synthetic construct
  source 1..40: mol_type = other DNA
atgaagctttcaacatggaatgggtccagcaagctcatga

SEQ ID NO: 30 - DNA, 40 bp, synthetic construct
  source 1..40: mol_type = other DNA
atgaagctttccatcaggaatgggtccagcaagctcatga

SEQ ID NO: 31 - DNA, 42 bp, synthetic construct
  source 1..42: mol_type = other DNA
atgaagctttcaacatcagagaatgggtcagcaggctcatga

SEQ ID NO: 32 - DNA, 60 bp, synthetic construct
  source 1..60: mol_type = other DNA
atgaagctttctcaggaatgggtcagcaggctcatgaaggaggagagaaaaagtgtttga

SEQ ID NO: 33 - RNA, 20 nt, synthetic construct
  source 1..20: mol_type = other RNA
acagttggtaggcacaccag